(19) United States
(12) Patent Application Publication
(10) Pub. No.: US 2026/0144880 A1
(43) Pub. Date: May 28, 2026

(54) SMALL MOLECULE LIGAND-TARGETED DRUG CONJUGATES FOR ANTI-INFLUENZA CHEMOTHERAPY AND IMMUNOTHERAPY

(71) Applicant: Purdue Research Foundation, West Lafayette, IN (US)

(72) Inventors: Philip S. LOW, West Lafayette, IN (US); Xin Liu, West Lafayette, IN (US)

(73) Assignee: Purdue Research Foundation, West Lafayette, IN (US)

(21) Appl. No.: 19/077,097

(22) Filed: Mar. 12, 2025

Related U.S. Application Data
(63) Continuation of application No. 17/263,451, now abandoned.
(60) Provisional application No. 62/703,534, filed on Jul. 26, 2018.
(60) Provisional application No. 62/846,549, filed on May 10, 2019.

(51) Int. Cl. A61K 47/55; A61K 47/54; A61K 49/00; A61K 51/04

(52) U.S. Cl. CPC A61K 47/55; A61K 47/54; A61K 47/542; A61K 49/0043; A61K 49/0052; A61K 51/0497

(57) ABSTRACT

Disclosed herein is a small molecule targeted drug conjugate for anti-influenza chemotherapy and immunotherapy. The disclosed drug conjugate may form an adaptor to recruit additional CAR T cells or other immune cells for precise elimination of influenza virus-infected cells in a subject. Concurrently administered antibodies or pre-existing immunity in influenza-virus infected subject works well with the targeted conjugate to eliminate virus infected cells, saving valuable time for rescuing late stage patients.

### FIELD OF INVENTION

This disclosure provides a targeted delivery of anti-influenza therapy. Particularly, a small molecule ligand that specifically binds to influenza virus is conjugated to a payload of drug to invoke either direct killing or immunomodulation of influenza virus infected cells.

### BACKGROUND

Caused by Influenza virus infection, the acute febrile respiratory disease influenza (also known as “flu”) is still one of the most life-threatening disseminated diseases. According to Influenza Fact Sheet released by World Health Organization (WHO), Influenza spreads worldwide in seasonal epidemics, resulting in about 3 to 5 million yearly cases of severe illness and about 250,000 to 500,000 yearly deaths.¹ In the united states, there are between 12,000 and 56,000 deaths and between 140,000 to 710,000 hospitalizations are directly associated with influenza per year 2 In addition to causing high morbidity and mortality, influenza imposes a substantial social economic burden arising from the productivity lost and medical prevention and treatment. The total annual cost associated with influenza has been over $10 billion in the U.S.³

### Current Anti-Influenza Chemotherapy for Influenza

Because influenza virus constantly changes via antigen shift and drift, vaccines often become ineffective against mutating strains. Therefore, anti-influenza chemotherapy still plays an important role in the prophylaxis and treatment of influenza.⁴ At present, two classes of Anti-influenza drugs, M2 ion channel inhibitor and neuraminidase inhibitor, have been approved by U.S. Food and Drug Administration (FDA). M2 ion channel inhibitors include amantadine and rimantadine. The mechanism of action of these drugs results from blocking the acid-activated viral M2 ion channel, and as a consequence inhibiting the release of viral ribonucleoprotein from virion to host cytosol.⁴ However, both H1N1 and H3N2 viruses currently circulating in humans are resistant to these inhibitors. Thus, Centers for Disease Control and Prevention (CDC) advises against their use due to the rapid emergence of drug resistance.⁵ The commonly used neuraminidase inhibitors include oseltamivir and zanamivir. They act as competitive inhibitors competing with sialic acid to bind to the active site of neuraminidase.⁴ While these inhibitors are effective against both influenza A and influenza B viruses, they have two major limitations. First, only small benefits were observed for neuraminidase inhibitors in terms of symptom severity alleviation and sickness duration reduction (0.6˜0.7 day out of 7 days).⁶ Second, this class of antivirals also suffer from the drug resistant problem. An increase in the number of oseltamivir-resistant strains has been noted since 2007 to 2008 season. In light of the limitations of the current anti-influenza chemotherapies, there is an urgent need to develop new anti-influenza drugs with novel mechanisms of action.⁷

### SUMMARY OF THE INVENTION

This disclosure provide a conjugate comprising a targeting ligand (TL) for an envelope protein of an influenza virus, a linker (L) and a payload of drug (D), wherein the TL is a molecule that binds to the envelope protein, the linker is covalently bound to both the D and the TL, and the D is an imaging agent, a therapeutic drug, an immune modulator or the combination thereof.

In some preferred embodiment the aforementioned linker comprises a spacer and a cleavable or noncleavable bridge between the TL and the D.

In some preferred embodiment the aforementioned envelope protein of the influenza virus is Neuraminidase (NA) or Hemagglutinin (HA).

In some preferred embodiment the aforementioned TL is zanamivir.

In some preferred embodiment the aforementioned TL is selected from the group consisting of oseltamivir, zanamivir, peramivir and laninamivir.

In some preferred embodiment, the aforementioned conjugate comprises an imaging agent used to quantify the intensity of the influenza infection.

In some preferred embodiment the aforementioned imaging agent comprises a chelation complex containing technetium-99m (^{99m}Tc).

In some preferred embodiment, the aforementioned conjugate has a binding affinity to the NA at about 1 nM to about 15 nM.

In some preferred embodiment the aforementioned D is selected from the group consisting of Tubulysin B hydrazide, pimodivir, Ozanimod and SN38.

In some preferred embodiment the aforementioned conjugate is one of the following;

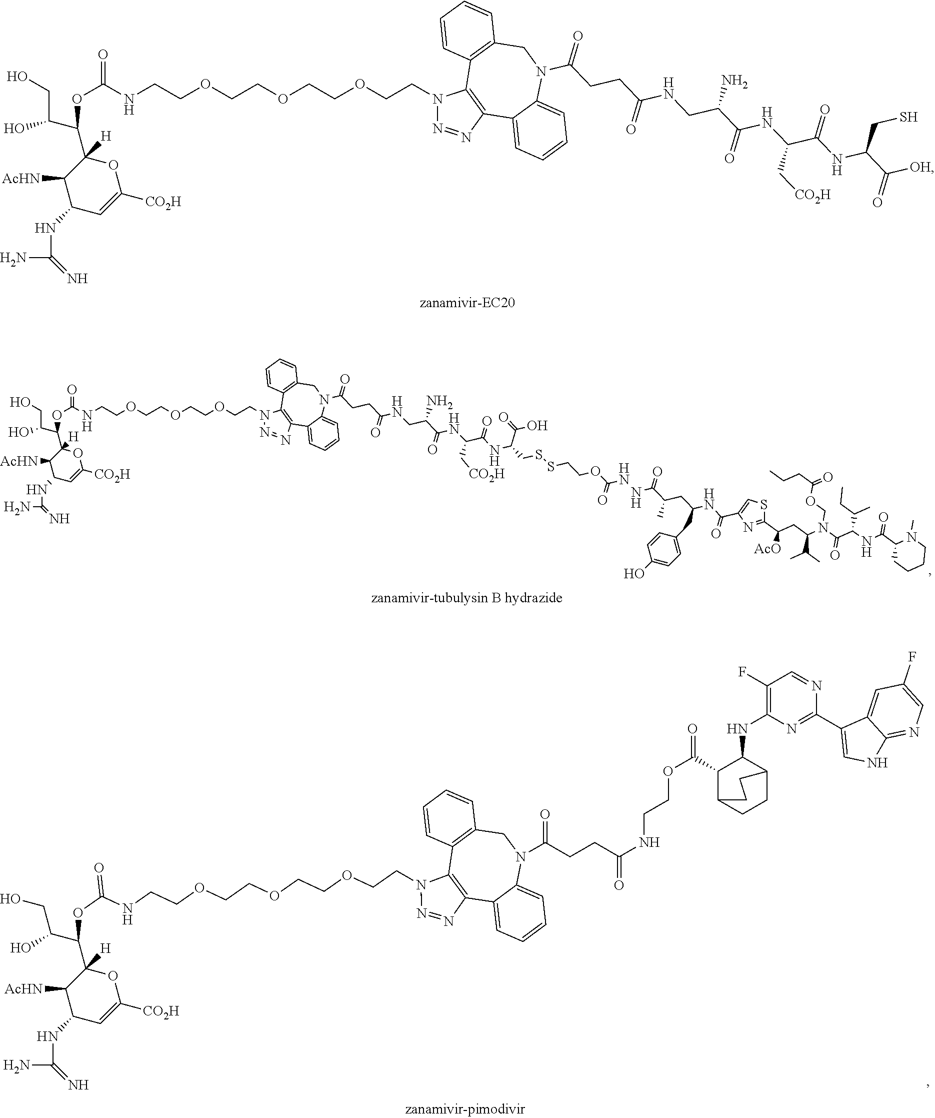

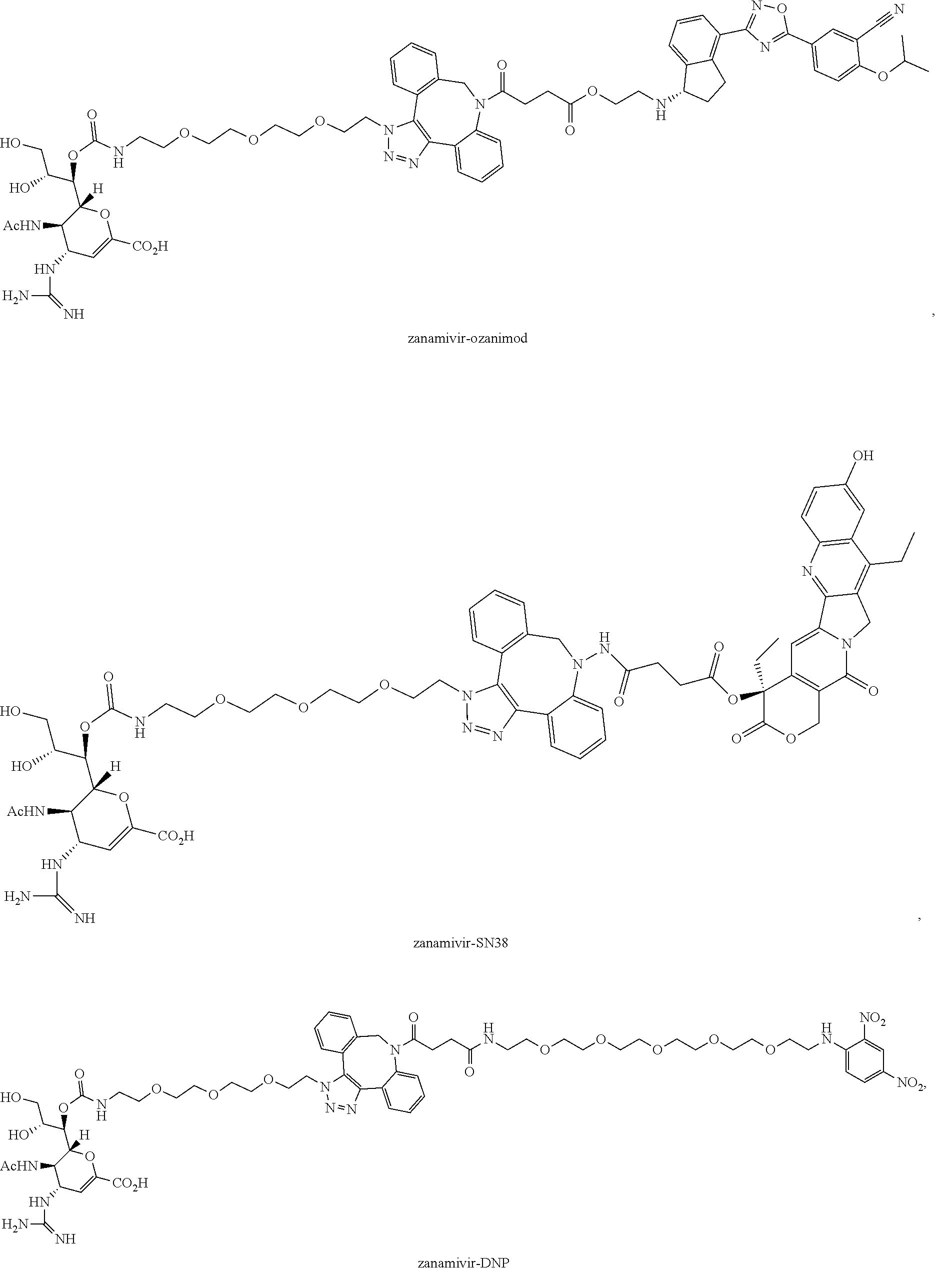

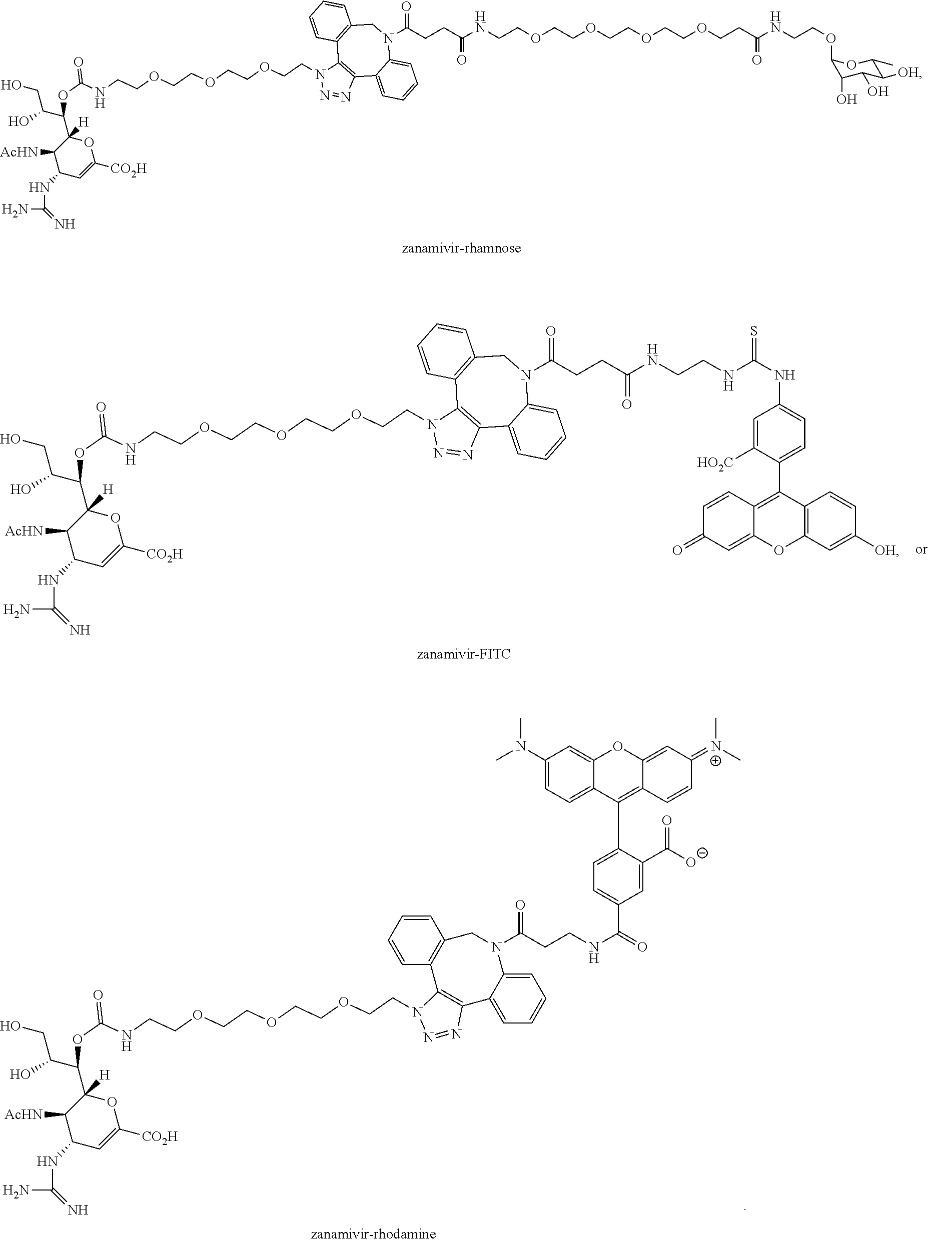

In some preferred embodiment the aforementioned cleavable bridge contains a disulfide or acid labile bond.

In some preferred embodiment the aforementioned acid labile bond comprises an ester, hydrazone, oxime, acetal, ketal, phenolic ether, or Schiff base bond.

This disclosure further provides a method to treat influenza virus infection in a subject, the method comprising providing a conjugate to the subject, wherein said conjugate comprises a targeting ligand (TL) of NA of the influenza virus, a linker (L) and a payload of drug (D), wherein the TL is a molecule that binds NA, the L is covalently bound to both the D and the TL, and the D is an imaging agent, a therapeutic drug, an immune modulator or the combination thereof.

In some preferred embodiment, the aforementioned method uses zanamivir as the TL.

In some preferred embodiment, the aforementioned method used therapeutic drug to kill influenza virus infected cells in the subject, or to inhibit influenza virus replication.

In some preferred embodiment, the aforementioned method uses therapeutic drug selected from the group consisting of Tubulysin B hydrazide, pimodivir, and SN38.

In some preferred embodiment, the aforementioned method used a therapeutic drug comprising an adaptor molecule (i.e. fluorescein covalently bound to the TL), and an anti-fluorescein CAR T cell, wherein upon binding to the adaptor molecule, said CAR-T cell kills influenza virus infected cell that expresses neuraminidase that binds with TL, and thereby inhibits influenza virus replication in the subject.

In some preferred embodiment, the aforementioned method used immune modulator to dampen influenza virus induced early cytokine storm.

In some preferred embodiment, the aforementioned method used immune modulator ozanimod or a hapten recognized by an autologous antibody.

In some preferred embodiment, the aforementioned hapten is comprised of dinitrophenyl (DNP), trinitrophenyl (TNP), rhamnose, or an alpha-galactosyl moiety.

In some preferred embodiment the aforementioned method used the zanamivir conjugate to elicit immune responses leading to the clearance of antibody-coated virus or virus infected cells via antibody dependent cellular phagocytosis (ADCP), antibody dependent cellular cytotoxicity (ADCC) or complement-dependent cytotoxicity (CDC).

In some preferred embodiment the aforementioned method used an antigen or another moiety to conjugate with zanamivir, wherein the subject has pre-existing immunity to the antigen or the moiety or the subject is concurrently administered with an effective dose of antibody to the antigen or the moiety. For example, this antigen or moiety can be a toxin (e.g. tetanus toxoid).

This disclosure further provides a system comprising at least two components, a first component comprising a conjugate containing a targeting ligand (TL) for an envelope protein of an influenza virus, a linker (L) and a payload of drug (D), wherein the TL is a molecule that binds the envelop protein, the L is covalently bound to both the D and the TL, and the D is a fluorescein; a second component comprising an anti-fluorescein CAR-T cell that binds the first component's fluorescein, wherein said system is promoted to kill an influenza virus-infected cell.

Representative zanamivir-DNP conjugate's in vitro binding assay has shown its high binding affinity for both N1 and N2 classes of neuraminidase. The conjugate is much more potent than zanamivir or oseltanmivir; it is effective even when added after the infection has developed much further in the patient; it can cure the infection with a single injection of our drug, and is effective against all strains of the flu.

These and other features, aspects and advantages of the present invention will become better understood with reference to the following figures, associated descriptions and claims.

### BRIEF DESCRIPTION OF THE DRAWINGS

FIG. 1 Action mechanism of zanamivir-therapeutic drug conjugate (a) schematic of the drug conjugate (b) proposed mechanism of action.

FIG. 2 Therapeutic drug payloads selected for zanamivir-targeted therapeutic drug conjugates.

FIG. 3 Action mechanism of zanamivir-hapten conjugate-targeted immunotherapy (a) schematic of zanamivir-DNP conjugate (b) proposed mechanism of action.

FIG. 4 Design of the targeting ligand based on zanamivir. The 7-OH group of zanamivir is highlighted in yellow.

FIG. 5 Crystal structure of zanamivir complexed with neuraminidase.

FIG. 6 Binding of zanamivir-rhodamine conjugate to influenza virus A/Puerto Rico/8/34 (H1N1) infected MDCK cells. (a) Confocal microscope images of drug group. Influenza virus infected MDCK cells incubated with 50 nM zanamivir-rhodamine conjugate; (b) Confocal microscope images of competition group. Influenza virus infected MDCK cells incubated with 50 nM zanamivir-rhodamine conjugate in the presence of 5 μM zanamivir; (c) Binding saturation curve.

FIG. 7 Binding of ^{99m}Tc chelated zanamivir-EC20 head conjugate to influenza virus A/Puerto Rico/8/34 (H1N1) infected MDCK cells. (a) Binding saturation curve; (b) Structure of ^{99m}Tc chelated zanamivir-EC20 head conjugate.

FIG. 8 Biodistribution of ^{99m}Tc chelated zanamivir-EC20 head conjugate in influenza virus A/Puerto Rico/8/1934 (H1N1) infected mice/uninfected mice.

FIG. 9 In vitro cytotoxicity of zanamivir-tubulysin B hydrazide conjugate and its component parts on neuraminidase transfected HEK 293 cells. The cytotoxicity of zanamivir-tubulysin B hydrazide conjugate (red circles), free tubulysin B hydrazide (orange triangles) and zanamivir-tubulysin B hydrazide conjugate in the presence of 100-fold excess of zanamivir (blue squares) are graphed.

FIG. 10 Competitive binding of zanamivir-DNP conjugate to neuraminidase transfected HEK 293 cells. (a) log (does)-response curve of zanamivir-DNP conjugate; (b) log (does)-response curve of zanamivir. zanamivir-rhodamine conjugate was used as the labelled ligand.

FIG. 11 Flow cytometry analysis demonstrating the ability of zanamivir-DNP conjugate to bind simultaneously to cell surface neuraminidase and antiDNP antibody. (a) Schematic depiction of flow cytometry-based antibody recruitment assay; (b) Flow cytometry analysis using neuraminidase transfected HEK293 cells (293tn NA); (c) Flow cytometry analysis using non-transfected HEK293 cells (293tn).

FIG. 12 In vivo protection efficacy of zanamivir-DNP conjugate against influenza virus A/Puerto Rico/8/1934 (H1N1) infection in BALB/c mice when administered 2 h after infection. (a) Body weight curve; (b) survival curve.

FIG. 13 proposed scheme of targeted CAR-T therapy for influenza infected cells.

FIGS. 14A and B. In vitro anti-FITC CAR-T killing profile with fluorescein adaptor-mediated FITC-zanamivir conjugates for cells expressing influenza surface protein NA. (A). CAR T: 293 NA=5:1 has 41% killing of 293NA cells; (B) CAR T: 293 NA=10:1 has 61% killing of 293NA cells.

FIG. 15. No binding of Zanamivir-FITC to normal 293T cells.

FIG. 16. The cytotoxicity against NA is specifically induced by Zanamivir-FITC.

FIG. 17. in vitro assay using real virus

FIG. 18. LDH assay of T cell Killing Influenza Infected MDCK versus CAR-T killing of influenza infected MDCK.

FIG. 19. Proposed mouse model for testing CAR T cell therapy of influenza-infected mouse.

FIGS. 20A-C. Mechanism of action anti-influenza immunotherapy includes: A. a small molecule ligand targeted drug conjugate: B. the structure of zanamivir-DNP conjugate: zanamivir (target ligand) is conjugated to Hapten (2,4-dinitrophneyl group) through a linker; C. upon the conjugate binding to the viral neuraminidase, the innate antibodies to DNP inhibits influenza virus replication. Thus the system redirects anti-dinitrophenyl (anti-DNP) antibodies to the influenza virus/virus-infected cells, induces the immune-mediated destruction of influenza virus/virus-infected cells.

FIGS. 21A-C. Various in-vitro binding assays in Influenza virus A/Puerto Rico/8/34 (H1N1) infected MDCK cells. A. Zanamivir-Rhodamine conjugate binding; B. free zanamivir binding and C. zanamivir-DNP-conjugate binding.

FIGS. 22A-C. Various in-vitro binding assays in Influenza virus A/Aichi/2/1968 (H3N2) infected MDCK cells. A. Zanamivir-Rhodamine conjugate binding; B. free zanamivir binding and C. zanamivir-DNP-conjugate binding.

FIGS. 23A and B. Anti-DNP antibody recruiting assay conducted in Influenza virus A/Puerto Rico/8/34 (H1N1) infected MDCK cells. A. assay flow chart, B. cell stain result from various conjugate addition (PE-red, TO-RPO-3 lolide-blue to show cell nucleus) and binding curve

FIGS. 24A and B. Anti-DNP antibody recruiting assay conducted in Influenza virus A/Aichi/2/1968 (H3N2) infected MDCK cells. A. assay flow chart; B. cell stain result from various conjugate addition (PE-red, TO-RPO-3 lolide-blue to show cell nucleus) and binding curve.

FIGS. 25A and B. Complement-dependent cytotoxicity assay (CDC). A. flow scheme of complement-dependent cytotoxicity assay conducted in NA transfected 293 cells. B. only 10 nM drug conjugate is needed to mediate the maximum cell killing.

FIGS. 26A and B. Antibody-dependent phagocytosis assay (ADCP). A. ADCP work flow. B. THP-1 cells (human macrophage) were treated with PMA and labeled with DiD before use. 293tnNA (GFP+) were incubated with THP-1 cells (ratio 1:1) with various concentrations of zanamivir-DNP conjugate, and anti-DNP antibody (100 nM) at 4° C. for 30 min. The ADCP effect was analysed by flow cytometry.

FIG. 27. Mouse protection study procedure: mice were immunized by subcutaneous injection of 2,4-Dinitrophenyl-Keyhole limpet Hemocyanin (DNP-KLH); Mice were infected with a lethal dose of influenza virus (100 LD50, A/Puerto Rico/8/1934 (H1N1)) at week 5; Treatment with zanamivir-DNP conjugate and other drugs starts after the infection and the mice were monitored for 2 weeks, Mice were counted as dead when losing either 25% of their initial weight or when they were moribund. B. Operation on a mouse.

FIGS. 28A and B. Dose escalation study (intranasal administration) Mice (5 mice/group) were infected with 50 uL H1N1 PR8 virus (100 LD₅₀, 4.2×10⁵ PFU) at day 0. Mice were intranasally given PBS/zanamivir/zanamivir-DNP conjugate 24 h post-infection, twice daily for five days. Mice were counted as dead when losing either 25% of their initial weight or when they were moribund. A. graphing of body weight percentage B. Percentage of survival mice according to the definition.

FIGS. 29A and B. Comparing the efficacy between zanamivir-DNP conjugates and its components (intranasal administration). Procedure: Mice (5 mice/group) were infected with 50 uL H1N1 PR8 virus (100 LD₅₀, 4.2×10⁵ PFU) at day 0. Mice were intranasally given zanamivir-DNP conjugate and its components 24 h post-infection, twice daily for five days. Mice were counted as dead when losing either 25% of their initial weight or when they were moribund. A. graphing of body weight percentage B. Percentage of survival mice according to the definition.

FIGS. 30A and B. Delayed-start-to-treat study (intranasal administration) procedure: DNP-KLH immunized mice were infected with 50 uL H1N1 PR8 virus (100 LD₅₀, 4.2×10⁵ PFU) at day 0. Mice were intranasally given 1.5 umol/kg zanamivir-DNP conjugate 48 h/72 h/96 h post-infection, twice daily for 7 days. Mice were counted as dead when losing either 25% of their initial weight or when they were moribund. A. graphing of body weight percentage B. Percentage of survival mice according to the definition.

FIGS. 31A and B. One dose treatment (intranasal administration) procedure: Mice (5 mice/group) were infected with 50 uL H1N1 PR8 virus (100 LD₅₀, 4.2×10⁵ PFU) at day 0. Mice were intranasally given PBS/zanamivir-DNP conjugate 24 h post-infection for only one time. Mice were counted as dead when losing either 25% of their initial weight or when they were moribund. A. graphing of body weight percentage B. Percentage of survival mice according to the definition.

FIGS. 32A-D. Bio-distribution and SPECT/CT imaging. A. new zanamivir-E20 head conjugate structure. B. cell bound radioactivity according to procedure below:
Mice were infected by influenza virus (H1N1) 3d before the experiment.
The mice were intravenous inject with 10 nmol zanamivir-EC20 head conjugate (150 μCi).
The radioactivity was counted 4 h post-injection.
   C. binding curve of zanamivir-E20 head in the presence of competitor 100×free zanamivir. D. SPECT/CT imaging post injection of conjugate without (left) and with 100× free zanmivir according to the procedure below:
Mice were infected by influenza virus (H1N1) 3d before the experiment.
The mice were intravenous injected with 50 nmol zanamivir-EC20 head conjugate (750 μCi).
The SPECT/CT imaging was performed 4 h post-injection

FIGS. 33A and B. Dose escalation study (intraperitoneally administration) according to the following procedure: Mice (5 mice/group) were infected with 50 uL H1N1 PR8 virus (100 LD₅₀, 4.2×10⁵ PFU) at day 0. Mice were intraperitoneally given PBS/zanamivir/zanamivir-DNP conjugate 24 h post-infection, twice daily for five days. Mice were counted as dead when losing either 25% of their initial weight or when they were moribund. A. graphing of body weight percentage B. Percentage of survival mice according to the definition.

FIGS. 34A and B. Comparing the efficacy between zanamivir-DNP conjugates and its components (intraperitoneally administration) according to the following procedure: Mice (5 mice/group) were infected with 50 uL H1N1 PR8 virus (100 LD⁵⁰, 4.2×10⁵ PFU) at day 0. Mice were intraperitoneally given PBS/zanamivir/zanamivir-DNP conjugate 24 h post-infection, twice daily for five days. Mice were counted as dead when losing either 25% of their initial weight or when they were moribund. A. graphing of body weight percentage B. Percentage of survival mice according to the definition.

FIGS. 35A and B. Antibody-dependent cellular toxicity assay (ADCC). A. The ADCC Reporter Bioassay uses engineered Jurkat cells stably expressing the FcγRIIIa receptor and an NFAT (nuclear factor of activated T-cells) response element driving expression of firefly luciferase as effector cells. Antibody biological activity in ADCC MOA is quantified through the luciferase produced as a result of NFAT pathway activation. B. ADCC work scheme and results for DNP-Zanamivir.

FIGS. 36A and B. In vitro antiviral assay for H1N1 and H3N2 infected MDCK cells. A. A/Puerto Rico/8/34 (H1N1) and B. A/Aichi/2/1968 (H3N2).

FIGS. 37A and B. Single dose treatment (intranasal administration) for H3N2 virus infected mice. A. Treatment effects measured by body weight maintenance. B. treatment effects measured by survival percentage.

FIGS. 38A and B. Single dose treatment (intraperitoneally administration) for H1N1 PR8 virus infected mice. A. Treatment effects measured by body weight maintenance. B. treatment effects measured by survival percentage.

FIGS. 39A and B. Single dose treatment (intraperitoneally administration) for H3N2 virus infected mice.
A. Treatment effects measured by body weight maintenance. B. treatment effects measured by survival percentage.

FIGS. 40A and B. Anti-DNP antibody and zana-DNP treated unimmunized mice infected by H1N1 PR8 virus. Unimmunized mice were intravenously given different doses of anti-DNP antibody one day after infected with lethal dose of H1N1 PR8 virus, and immediately treated by single dose of intraperitoneally administered zanamivir-DNP conjugate. A. Treatment effects measured by body weight maintenance. B. treatment effects measured by survival percentage.

FIG. 41. Synthesis scheme of zanamivir-rhamnose conjugate, a different conjugate that utilizes innate immune system produced anti-rhamnose to markup influenza virus infected cells and induce immune attacks to influenza viruses infected cells.

FIGS. 42A and B. Competitive binding of zanamivir-DNP rhamnose conjugate to neuraminidase transfected HEK293 cells using zanamivir-rhodamine conjugate as the labelled ligand. A. Zanamivir Kd about 0.77 nM. B. Zanamivir-rhamnose Kd about 3.57 nM.

FIGS. 43A and B. Immunotherapy study with zanamivir-rhamnose conjugate. Rhamnose-OVA immunized mice (5 mice/group) were infected with 50 uL H1N1 PR8 virus (100 LD₅₀, 4.2×10⁵ PFU) at day 0. Mice were intranasally given 1.5/0.5/0.17 umol/kg zanamivir-rhamnose conjugate/zanamivir/PBS 24 h post-infection, twice daily for 5 days and mice were counted as dead when losing either 25% of their initial weight or when they were moribund. A. Treatment effects measured by body weight maintenance. B. treatment effects measured by survival percentage.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

### Influenza Virus in General

Influenza virus is an enveloped virus. All influenza subtypes are very similar in overall structure. The virus particle is 80-120 nanometers in diameter and usually roughly spherical, although filamentous forms can occur. These filamentous forms are more common in influenza C, which can form cordlike structures up to 500 micrometers long on the surfaces of infected cells. However, despite these varied shapes, the viral particles of all influenza viruses are similar in composition. These are made of a viral envelope containing two main types of glycoproteins, wrapped around a central core. The central core contains the viral RNA genome and other viral proteins that package and protect this RNA. RNA tends to be single stranded but in special cases, it is double stranded. Unusually for a virus, its genome is not a single piece of nucleic acid; instead, it contains seven or eight pieces of segmented negative-sense RNA, each piece of RNA containing either one or two genes, which code for a gene product (protein). For example, the influenza A genome contains 11 genes on eight pieces of RNA, encoding for 11 proteins: hemagglutinin (HA), neuraminidase (NA), nucleoprotein (NP), M1, M2, NS1, NS2 (NEP: nuclear export protein), PA, PB1 (polymerase basic 1), PB1-F2 and PB2.

Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1. There are 16 H and 9 N subtypes known, but only H 1, 2 and 3, and N 1 and 2 are commonly found in humans.

### Small Molecule Ligand-Targeted Drug Conjugate for Antiviral Therapy

Small molecule ligand-targeted drug conjugate, which combines the receptor-specific ligand with therapeutic payload, has shown promise in the treatment of many diseases especially in cancer chemotherapy. By specifically delivering the therapeutic payload to cells that are recognized by targeting ligand, these drug conjugates demonstrate high selectivity toward malignant cells as well as reduced associated collateral toxicity. To date, many cancers have been tackled by small molecule ligand-targeted drug conjugates that target overexpressing receptors on tumor cells. These overexpressing receptors include folate receptor (FR), prostate-specific membrane antigen (PSMA), cholecystokinin 2 receptor (CCK2R), carbonic anhydrase IX (CA IX), etc.

For enveloped virus, the last step of its replication involves assembling of viral components on the infected cell membrane and budding from the infected cell surface.⁹ Meanwhile, some virus envelope glycoproteins, such as HIV gp120 and influenza neuraminidase/hemagglutinin, are expressed on the exterior surface of infected cells.¹⁰ In light of the fact that these exogenous viral proteins are exclusively expressed on the infected cells, they have the potential to be targeted by ligand targeted drug conjugates.

### Design of Zanamivir-Therapeutic Drug Conjugates

The general scheme of the instant disclosure is to provide a specific targeting ligand conjugated to an effective payload of therapeutic drug or modulator to treat virus infections. The targeting ligand will specifically recognize the envelop protein of the virus, which is exclusively expressed on the surface of the infected cells. In some occasions, the payload of therapeutic drug or modulator can be an adapted chimeric antigen receptor-expressing T cell (CAR T cell). For example, if the payload of drug is a fluorescein adaptor, an anti-fluorescein CAR T cell can be administered along with the targeted ligand guided payload drug to either kill the virus infected cells, or inhibit the replication of the virus in the infected cells. For a detailed description of an adaptor molecule-mediated CAR T cell therapy and its makes thereof, please see U.S. application Ser. No. 15/296,666, filed on Oct. 16, 2016, the entire contents of which is incorporated herein by reference.

Here we designed a series of small molecule ligand targeted drug conjugates targeting influenza virus envelop protein, particularly neuraminidase (NA). Without being bound by any theory, it is contemplated that other small molecule ligands that specifically target Hemagglutinin (HA) may also work under this principle. For example, compounds that inhibit HA mediated influenza virus entry may be considered as potential targeting ligands effecting on HA of infected cells.

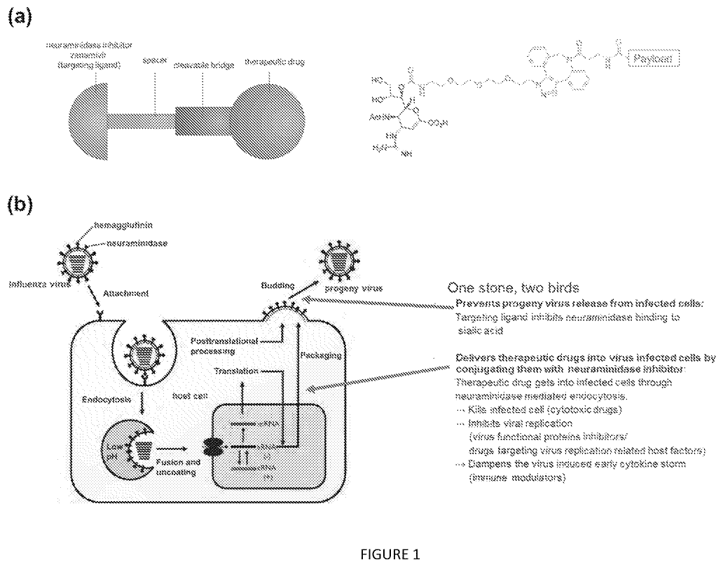

Accordingly, high affinity neuraminidase inhibitor zanamivir was herein repurposed to carry and deliver the therapeutic drugs specifically into the virus infected cells as well as the virus replication sites (e.g. nose, throat, and lungs). This presents a unique mechanism of action by which one can kill the virus infected cells prior to the progeny virus release, hinder the viral replication, or dampen the early cytokine storm induced by the virus infection (FIG. 1).

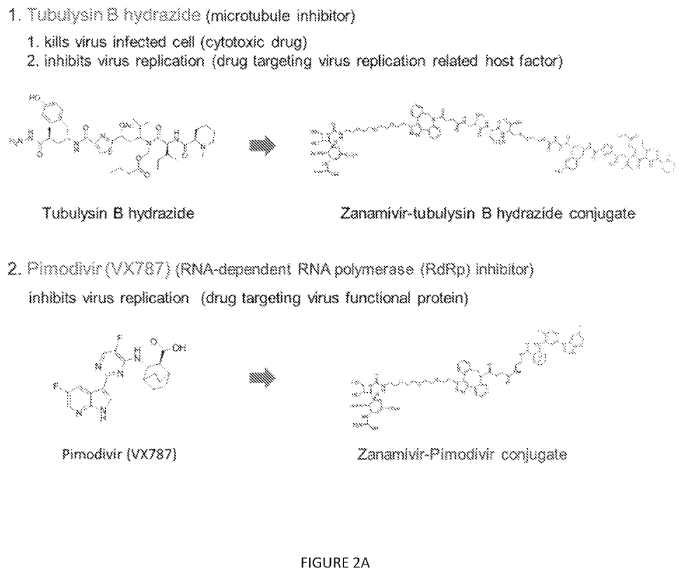

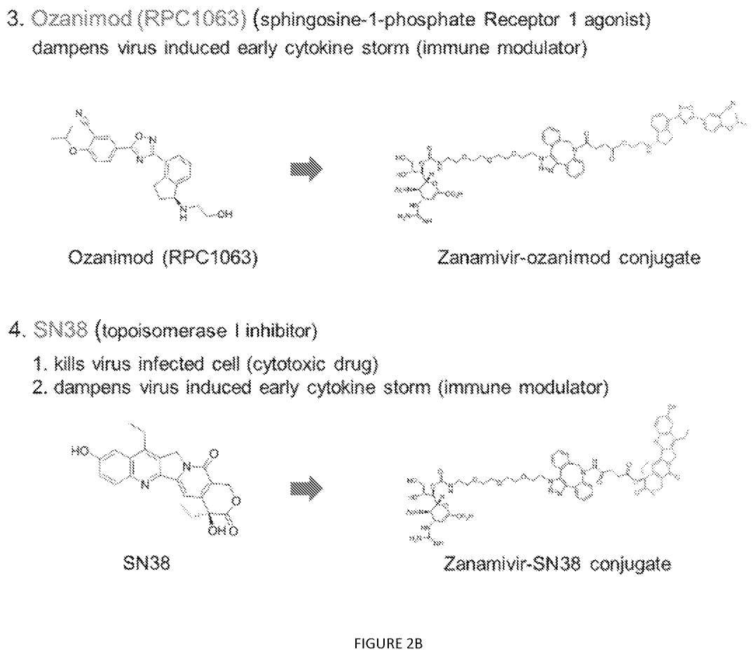

The therapeutic drug payloads selected for this project are shown in FIG. 2 (1) Tubulysin B hydrazide is an antimitotic tetrapeptide that inhibits tubulin polymerization. It either kills the influenza virus infected cells by inducing cell apoptosis, or inhibits the transportation of viral components by destructing the microtubule network of influenza virus infected cells.¹¹ (2) Pimodivir is a RNA-dependent RNA polymerase (RdRp) inhibitor that blocks m⁷GTP binding pocket in the PB2 subunit of influenza A viral polymerase complex. It interferes with virus replication by inhibiting PB2 cap-snatching activity.^{12, 13} In view of the fact that the high morbidity and mortality caused by influenza are the result of both virus induced tissue destruction and hyper-induction of proinflammatory cytokine production (cytokine storm), two immunomodulatory drugs, Ozanimod and SN38, are selected in order to improve the outcome of influenza treatment.^{14, 15} (3) Ozanimod is an investigational immunomodulatory drug acting as a sphingosine-1-phosphate (SIP) receptor agonist.¹⁶ Researchers found that S1P receptor agonists can blunt but not abolish the excess virus induced cytokine production, providing significant protection against influenza virus infection in mice.^{17, 18} (4) SN38 is a topoisomerase I inhibitor, which is the active metabolite of irinotecan (an analog of camptothecin).¹⁹ SN 38 was demonstrated to limit the overexpression of influenza virus induced inflammatory genes through inhibiting the recruitment of RNA polymerase II to innate immune genes.²⁰ In addition, SN38 can also kill the influenza virus infected cells by inducing cell apoptosis.

When these molecules including but not limited to Tubulysin B hydrazide, Pimodivir, Ozanimod, or SN38 are conjugated with the targeting ligand of virus envelop protein, they can play various roles of killing virus infected cells, or inhibiting the virus replication within the infected cells

### Design of Zanamivir-Hapten Conjugate-Targeted Immunotherapy for the Treatment of Influenza

Other than therapeutic drugs that can directly kill influenza virus infected cells or inhibit the replication of the virus within infected cells, immunotherapy can be effective to elicit immune system to fight the specific infection by antibodies existing in the body. One possible candidate for such immunotherapy is to wake up the circulating anti-DNP antibodies by making a conjugate of TL with dinitrophenyl (DNP).

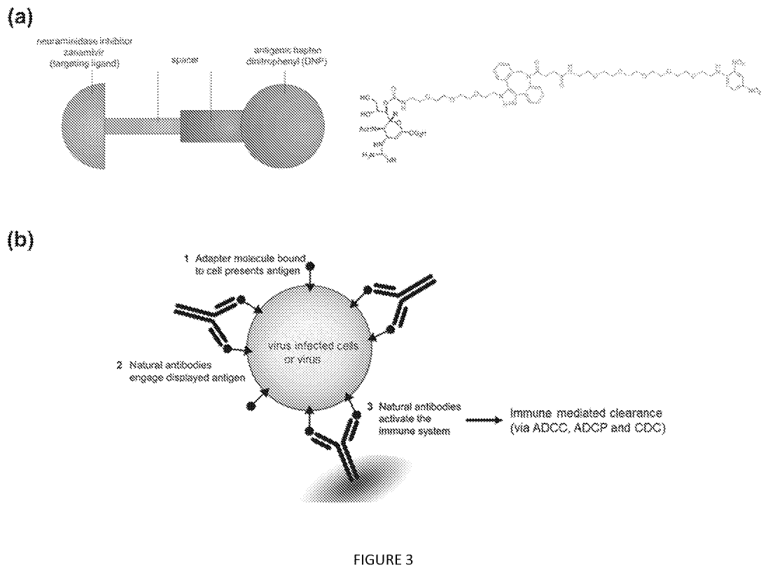

Because of the potential targeting ability of zanamivir to influenza virus or virus infected cells, a zanamivir-dinitrophenyl (DNP) conjugate was also developed in our lab (FIG. 3). As shown in FIG. 3b, zanamivir-DNP conjugate is believed to form a bispecific molecular “bridge” between influenza virus/virus infected cells and endogenous circulating anti-DNP antibodies. This “marking” step initiates the immune response leading to the clearance of the antibody-coated virus or virus infected cell via mechanisms such as antibody-dependent cellular phagocytosis (ADCP), antibody-dependent cellular cytotoxicity (ADCC) and complement-dependent cytotoxicity (CDC).²¹⁻²³

There are two major advantages of zanamivir-DNP conjugate-targeted immunotherapy over influenza vaccines. First, current influenza vaccines are prepared based on predicting which subtypes of the virus will likely appear in the next season. Because this prediction occasionally fails, the vaccines can not precisely match the virus. However, zanamivir is effective for all 11 influenza NA subtypes with high affinities, and there are very few zanamivir resistant viruses are found in the clinic.⁷ Second, since anti-DNP antibodies are already present in the human bloodstream, the pre-vaccination is not necessary for this therapy.²⁴

Without being bound by any theory, it is contemplated that zanamivir conjugated to any other moieties such as trinitrophenyl (TNP), rhamnose, or an alpha-galactosyl may recruit their respective antibodies to the influenza infected cells to elicit antibody-dependent immune responses. Thus, immunotherapy disclosed herein can target influenza virus infected cells that by zanamivir conjugate markings.

### Influenza Virus Induced Tumor Types for Targeted CAR T Cell Therapy

In connection with our newly developed adapted CAR T cell therapy, as described in US application Ser. No. 15/296,666 or its related applications (the contents of which are expressly incorporated herein by reference), targeted delivery of CAR T cells to influenza virus infected cells to execute CAR T cells immune response functions is contemplated in this disclosure.

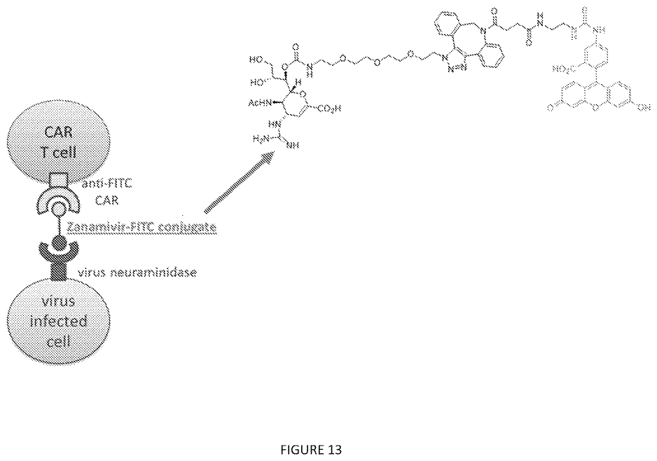

FIG. 13 depicts the CAR T cell strategy treating influenza virus infected cells. In FIG. 13, a zanamivir-FITC conjugate is produced and attached to an influenza virus-infected cell that expresses virus neuraminidase on the surface. It is noted that zanamivir-FITC may serve at least two different functions in this process, one is to serve as an imaging agent to illustrate the infection intensity of influenza virus; the other is to mark the virus-infected cells with zanamivir, an NA inhibitor that can block the virus budding from the envelop. The presence of zanamivir-FITC conjugate at the infected cell surface may direct a T cell adapted with anti-FITC antibody to virus infected cell and form immunological synapse. As a person skill in the art may know, such anti-FITC CAR T cells can be activated by the binding to zanamivir-FITC conjugate. Activated CAR T cell therefore may secret cytokines and subsequently kill virus-infected cells, preventing virus replication.

Without being limited by any theory, the advantages of using small molecule targeted drug or immune regulator conjugate to treat influenza infected cells can be seen from multiple facets. Currently vaccines are manufactured based on annual predicting of which strains will likely circulate during the next season. However, such strategy occasionally fails and thus causes vaccines ineffective for the predominant strain of virus in epidemic. The exemplified zanamivir, which is an NA inhibitor effective for all 11 influenza NA subtypes, blocks the virus budding from the infected cells. Thus the effectivity suits for all subtypes of influenza virus. At the same time, zanamivir conjugated payload drug, either a therapeutic agent, or an immunotherapy modulator, or an adaptor molecule (i.e. fluorescein) mediated anti-fluorescein CAR T cell, specifically mark influenza infected cells to elicit necessary immune response to clear the virus infected cells.

### EXAMPLES

### Example 1. Design of the Targeting Ligand

Influenza neuraminidase (NA) is a transmembrane glycoprotein anchored in the lipid raft domain of influenza virus envelope NA accounts for 20% (about 80) of the membrane glycoproteins and the head of NA is a homo-tetramer. It assists in the release of progeny virus from the infected cells by cleaving sialic acids from membrane glycoproteins or glycolipids (In the virus budding process, influenza virus hemagglutinin can bind to sialic acid receptors on the host cell membrane, which hinders the release of newly formed virus.).⁹ Since neuraminidases are expressed on both the influenza virus surface and the surface of infected cell membrane, it was selected by our group as the potential target for the design of targeting ligand to target influenza virus and virus infected cells.

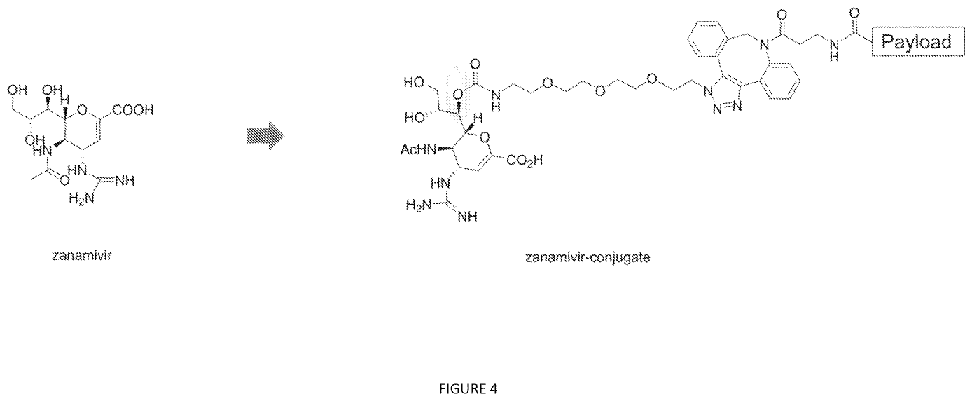

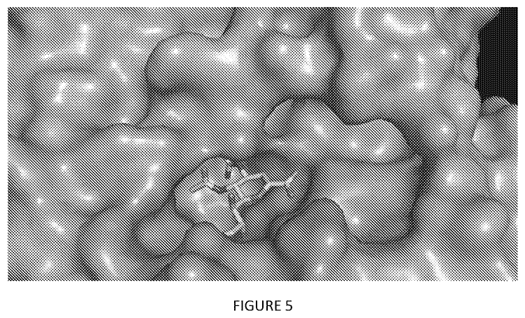

To date, four neuraminidase inhibitors have been developed as anti-influenza drugs: oseltamivir (Tamiflu; Glide/Roche), zanamivir (Relenza; GlaxoSmithKline), peramivir (Rapivab; BioCryst) and laninamivir (Inavir; Daiichi Sankyo).²⁵ Because zanamivir is an inhibitor derived from the naturally occurring sialic acid with minimal functionalization, rare zanamivir-resistant virus is found in the clinic.⁷ Therefore, zanamivir was selected as the candidate for the targeting ligand design among neuraminidase inhibitors. Honda et al. reported that C-7 alkyl-modified analogues of zanamivir retained their inhibitory activities against neuraminidase (FIG. 4), which indicates that the C-7 position is tolerant to be modified as the linker attachment site.²⁶ Honda's conclusion is supported by the result of X-ray crystallographic structure of zanamivir complexed with neuraminidase: the 7-OH group of zanamivir is exposed to the solvent surface area and makes no direct interaction with the active site of neuraminidase (FIG. 5).²⁷ Moreover, several research groups also used the C7 position as the linkage site to build a set of multimeric analogues of zanamivir and zanamivir derivatives with improved anti-influenza activity.²⁸ Taken together, we design a new NA targeting ligand based on zanamivir by modifying its 7-OH group as the linkage site (FIG. 4).

### Example 2. Compound Synthesis

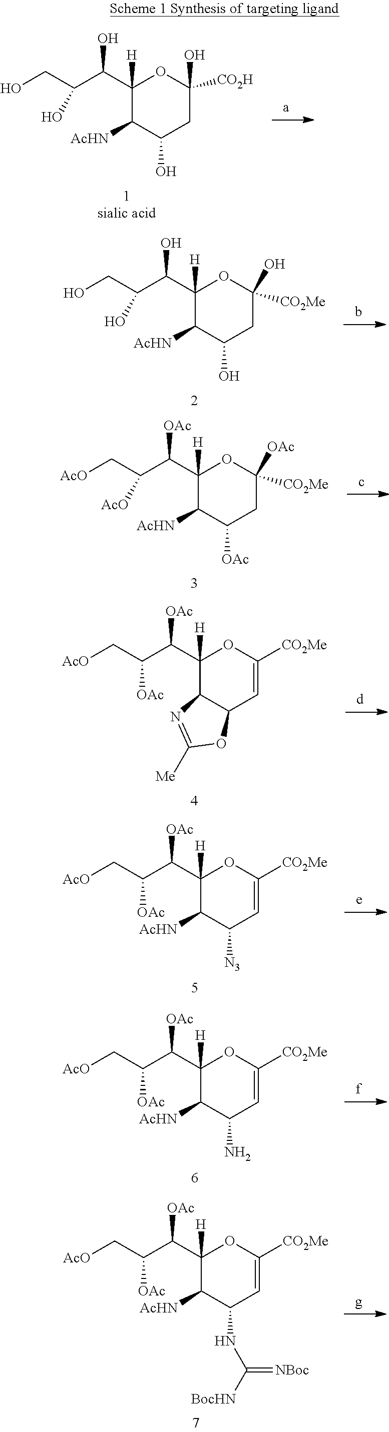

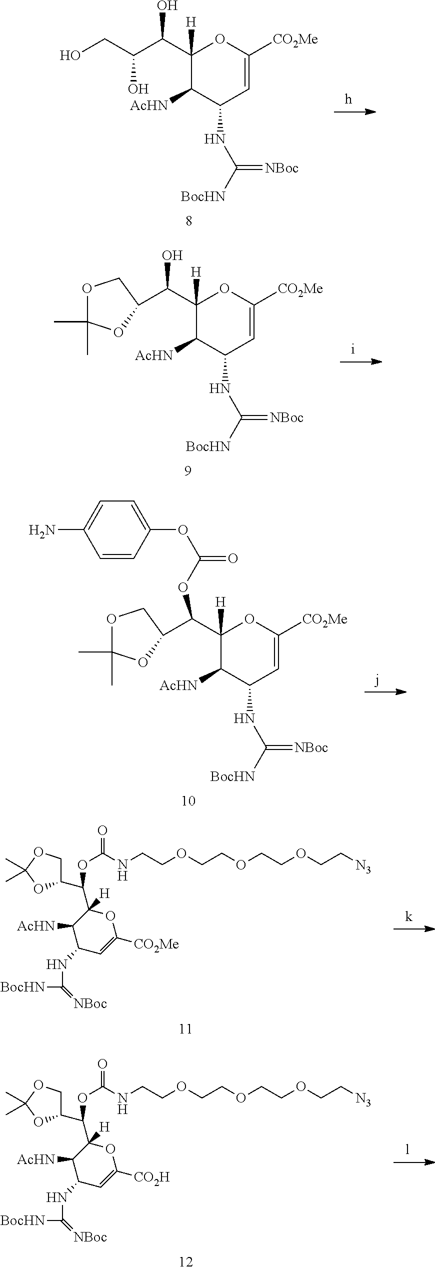

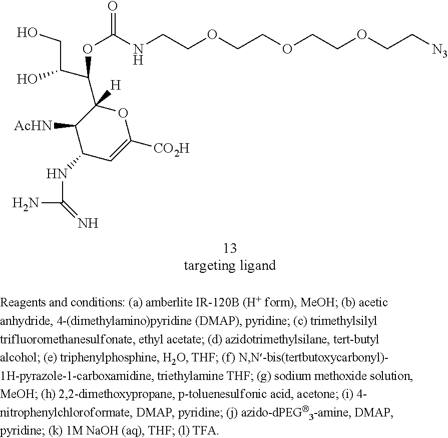 [SMILES: CC(=O)N[C@H]1[C@H]([C@H](OC(=O)NCCOCCOCCOCCN=[N+]=[N-])[C@H](O)CO)OC(C(=O)O)=C[C@@H]1NC(=N)N]

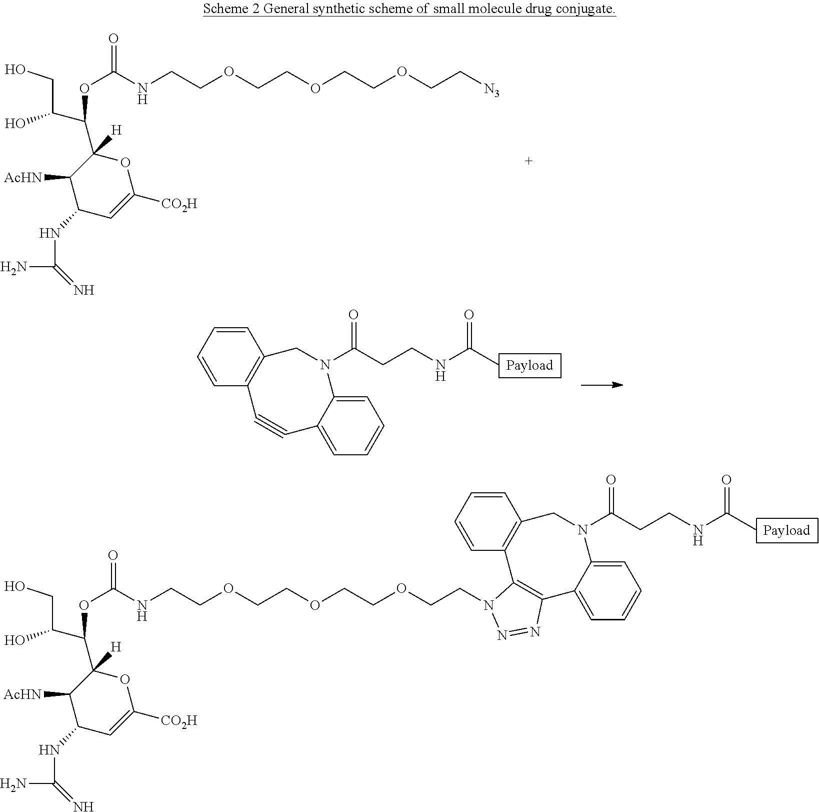 [SMILES: CC(=O)N[C@H]1[C@H]([C@H](OC(=O)NCCOCCOCCOCCN=[N+]=[N-])[C@H](O)CO)OC(C(=O)O)=C[C@@H]1NC(=N)N ; CC(=O)NCCC(=O)N1Cc2ccccc2C#Cc2ccccc21 ; CC(=O)NCCC(=O)N1Cc2ccccc2-c2c(nnn2CCOCCOCCOCCNC(=O)O[C@@H]([C@@H]2OC(C(=O)O)=C[C@H](NC(=N)N)[C@H]2NC(C)=O)[C@H](O)CO)-c2ccccc21]

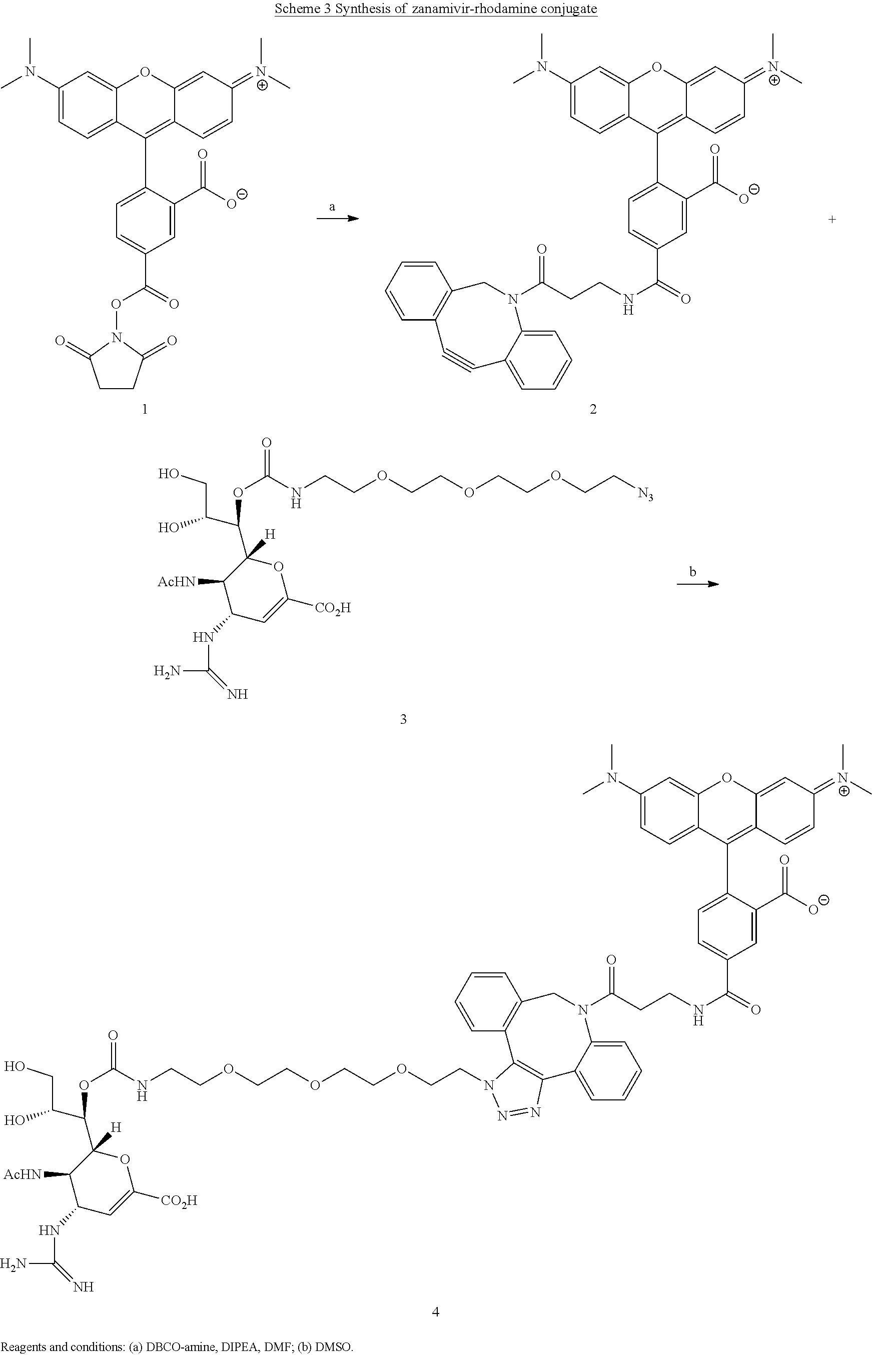

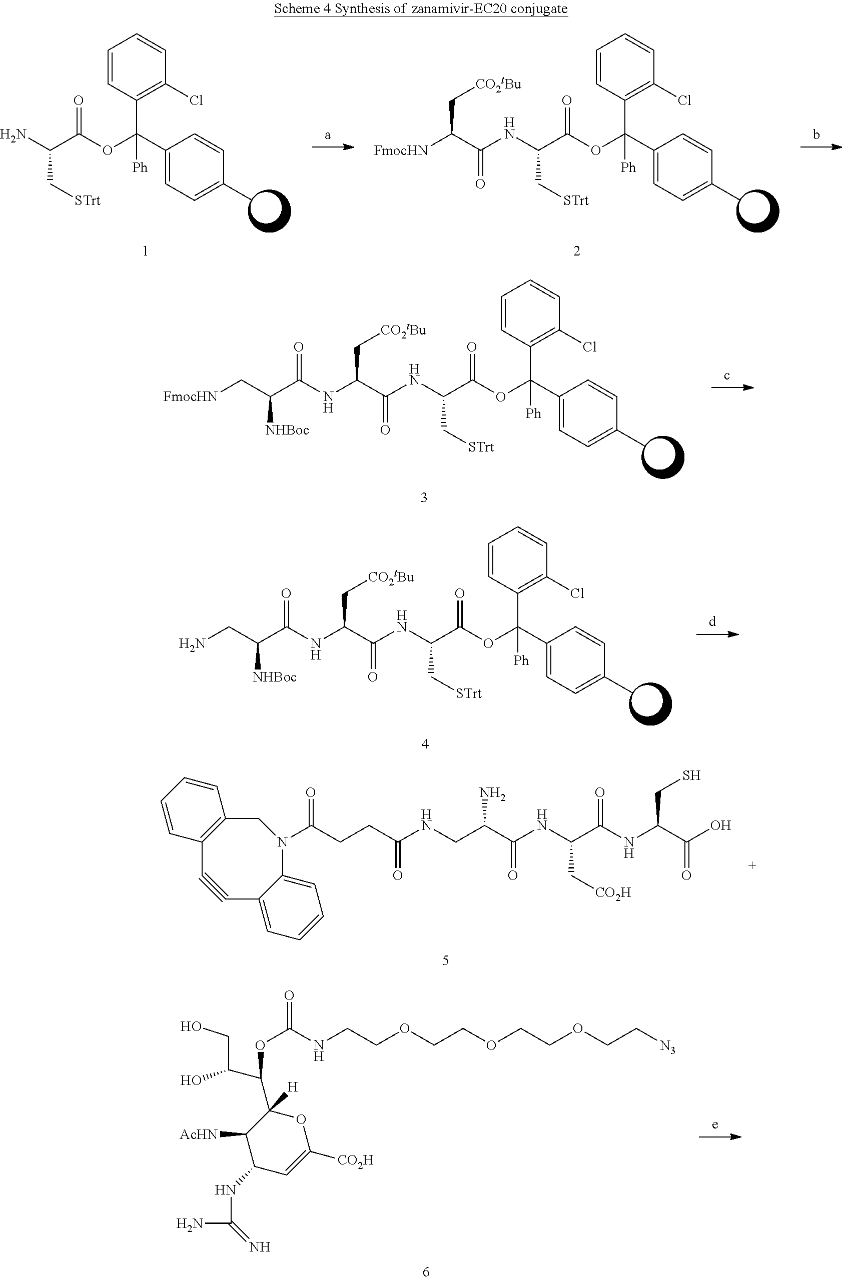

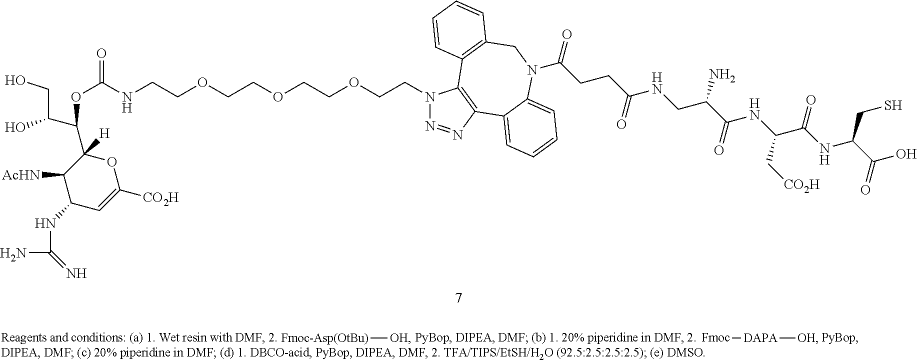

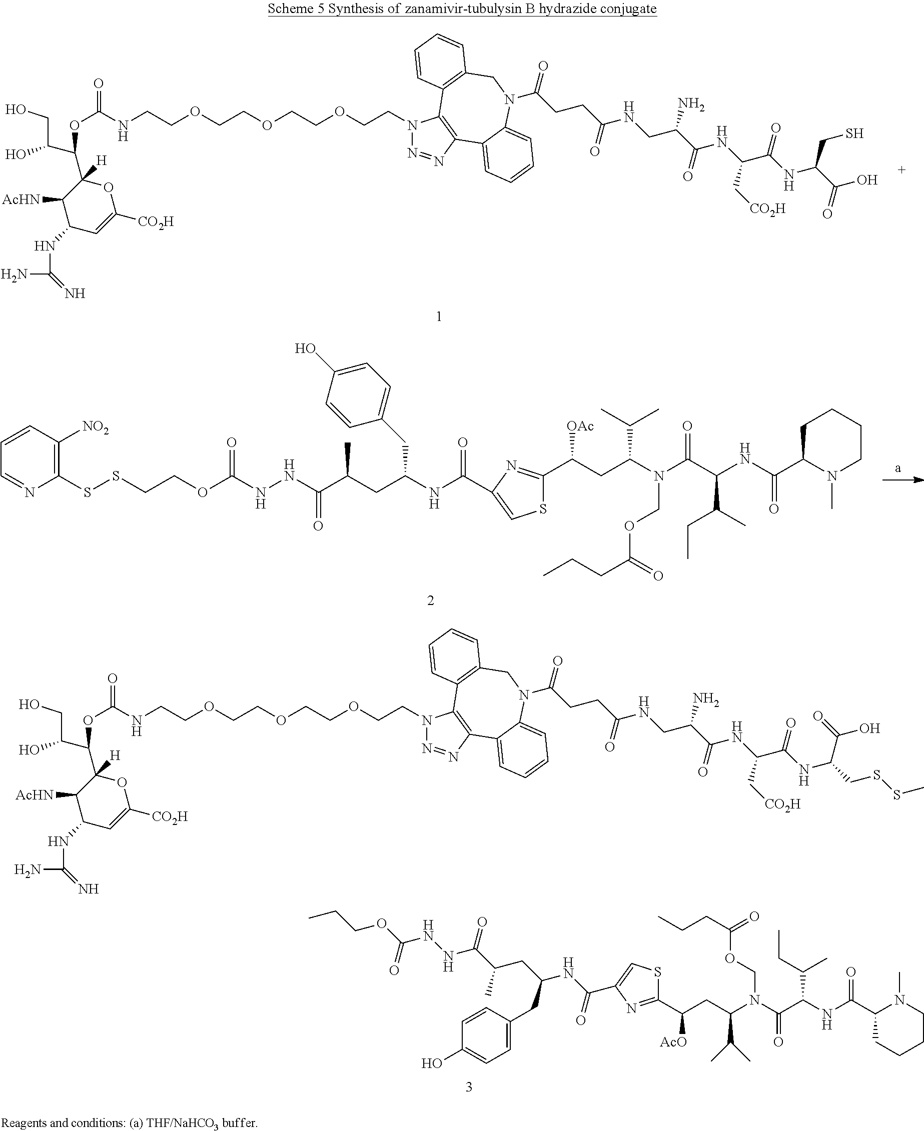

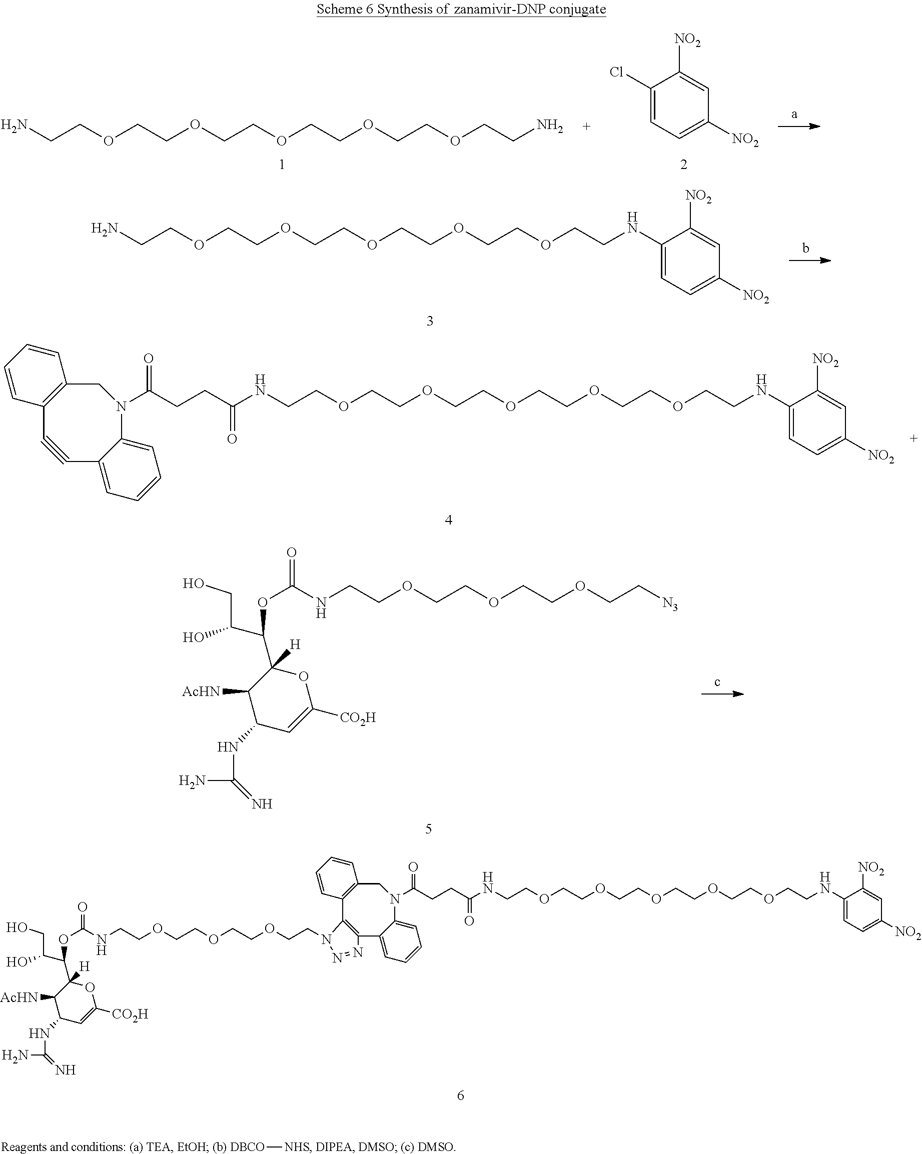

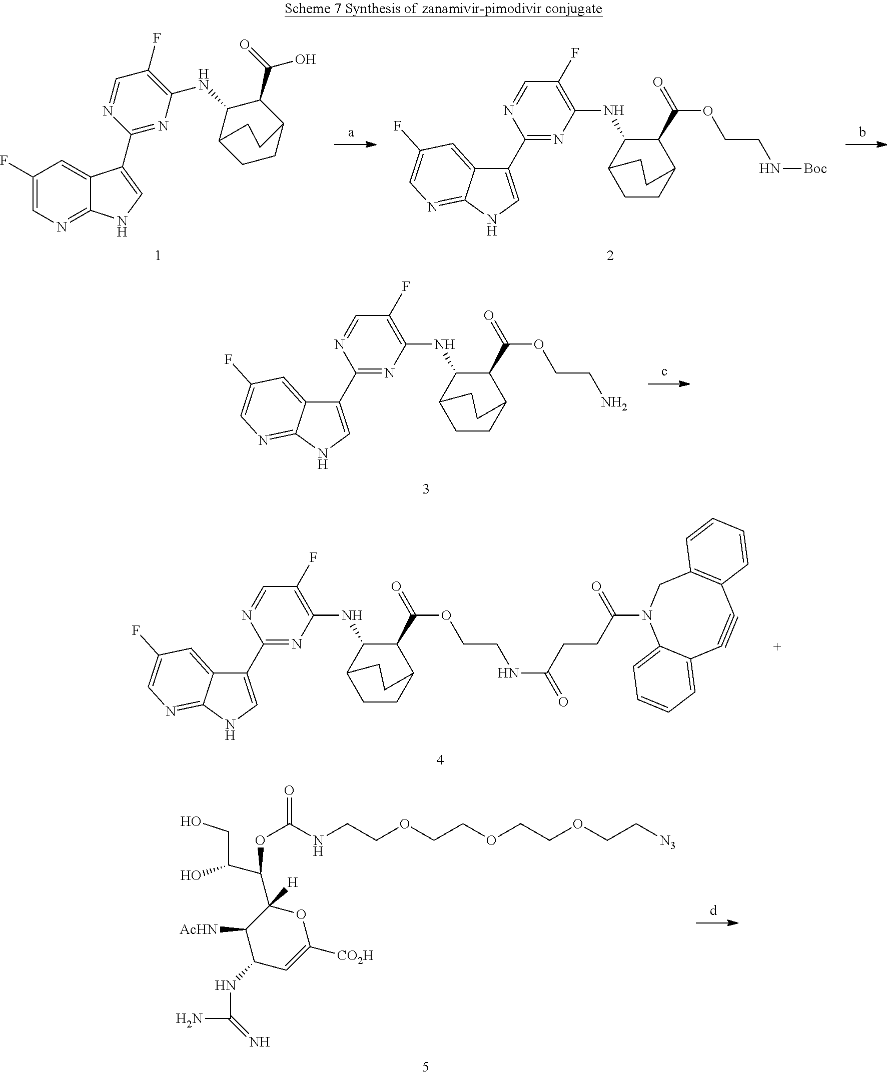 [SMILES: O=C(O)[C@H]1C2CCC(CC2)[C@@H]1Nc1nc(-c2c[nH]c3ncc(F)cc23)ncc1F ; CC(C)(C)OC(=O)NCCOC(=O)[C@H]1C2CCC(CC2)[C@@H]1Nc1nc(-c2c[nH]c3ncc(F)cc23)ncc1F ; NCCOC(=O)[C@H]1C2CCC(CC2)[C@@H]1Nc1nc(-c2c[nH]c3ncc(F)cc23)ncc1F ; O=C(CCC(=O)N1Cc2ccccc2C#Cc2ccccc21)NCCOC(=O)[C@H]1C2CCC(CC2)[C@@H]1Nc1nc(-c2c[nH]c3ncc(F)cc23)ncc1F ; CC(=O)N[C@H]1[C@H]([C@H](OC(=O)NCCOCCOCCOCCN=[N+]=[N-])[C@H](O)CO)OC(C(=O)O)=C[C@@H]1NC(=N)N]

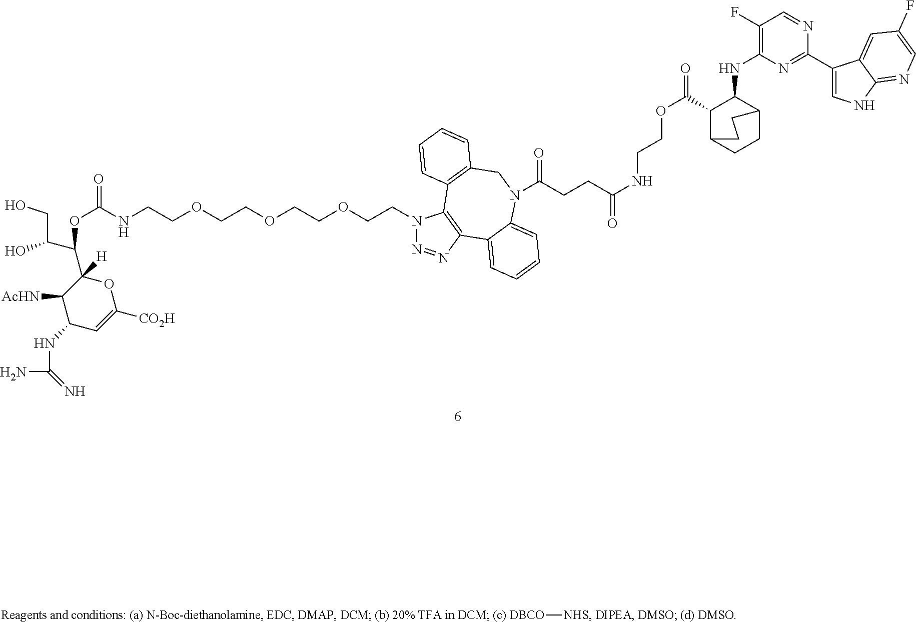

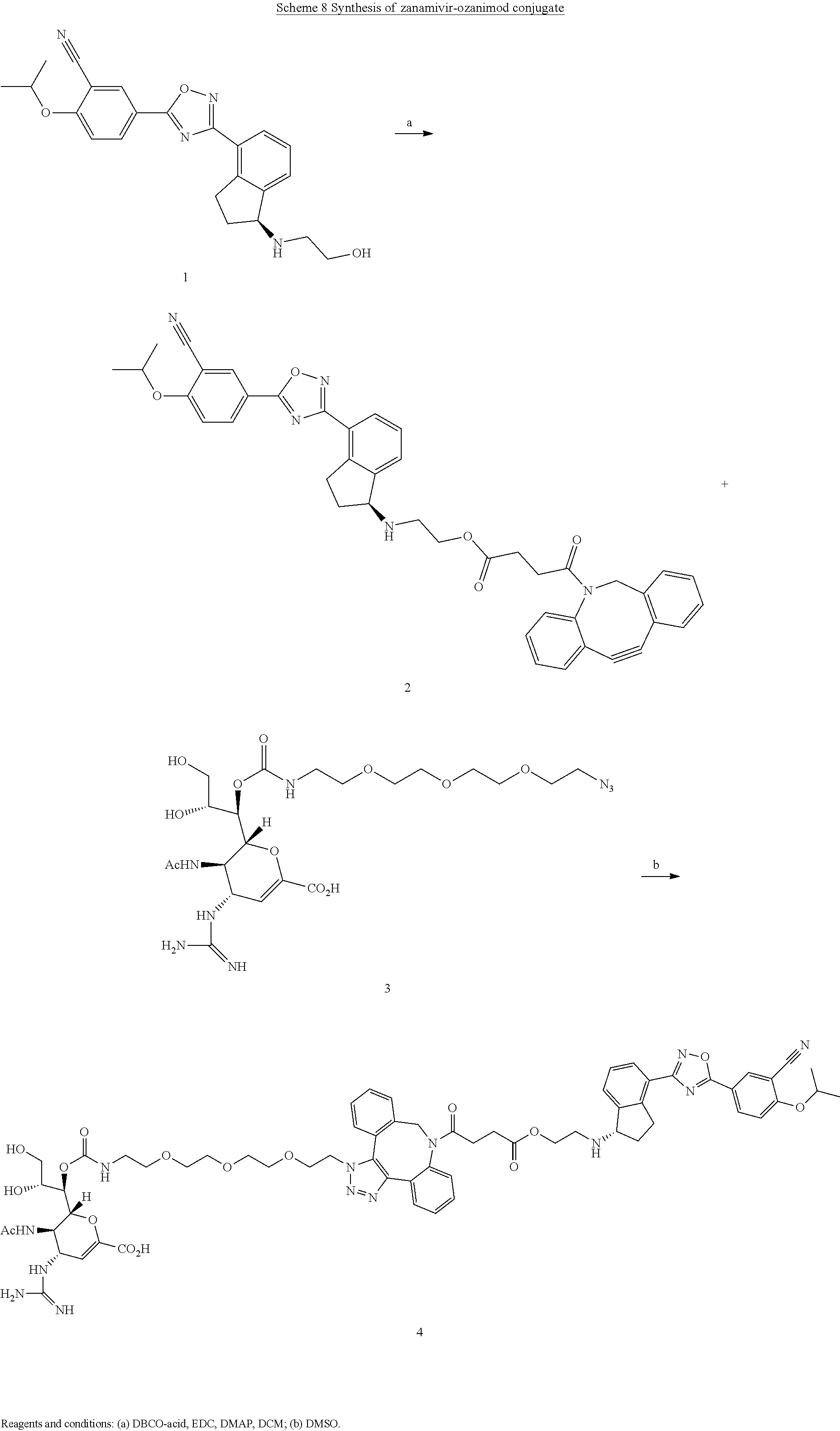 [SMILES: CC(C)Oc1ccc(-c2nc(-c3cccc4c3CC[C@@H]4NCCO)no2)cc1C#N ; CC(C)Oc1ccc(-c2nc(-c3cccc4c3CC[C@@H]4NCCOC(=O)CCC(=O)N3Cc4ccccc4C#Cc4ccccc43)no2)cc1C#N ; CC(=O)N[C@H]1[C@H]([C@H](OC(=O)NCCOCCOCCOCCN=[N+]=[N-])[C@H](O)CO)OC(C(=O)O)=C[C@@H]1NC(=N)N ; CC(=O)N[C@H]1[C@H]([C@H](OC(=O)NCCOCCOCCOCCn2nnc3c2-c2ccccc2CN(C(=O)CCC(=O)OCCN[C@H]2CCc4c(-c5noc(-c6ccc(OC(C)C)c(C#N)c6)n5)cccc42)c2ccccc2-3)[C@H](O)CO)OC(C(=O)O)=C[C@@H]1NC(=N)N]

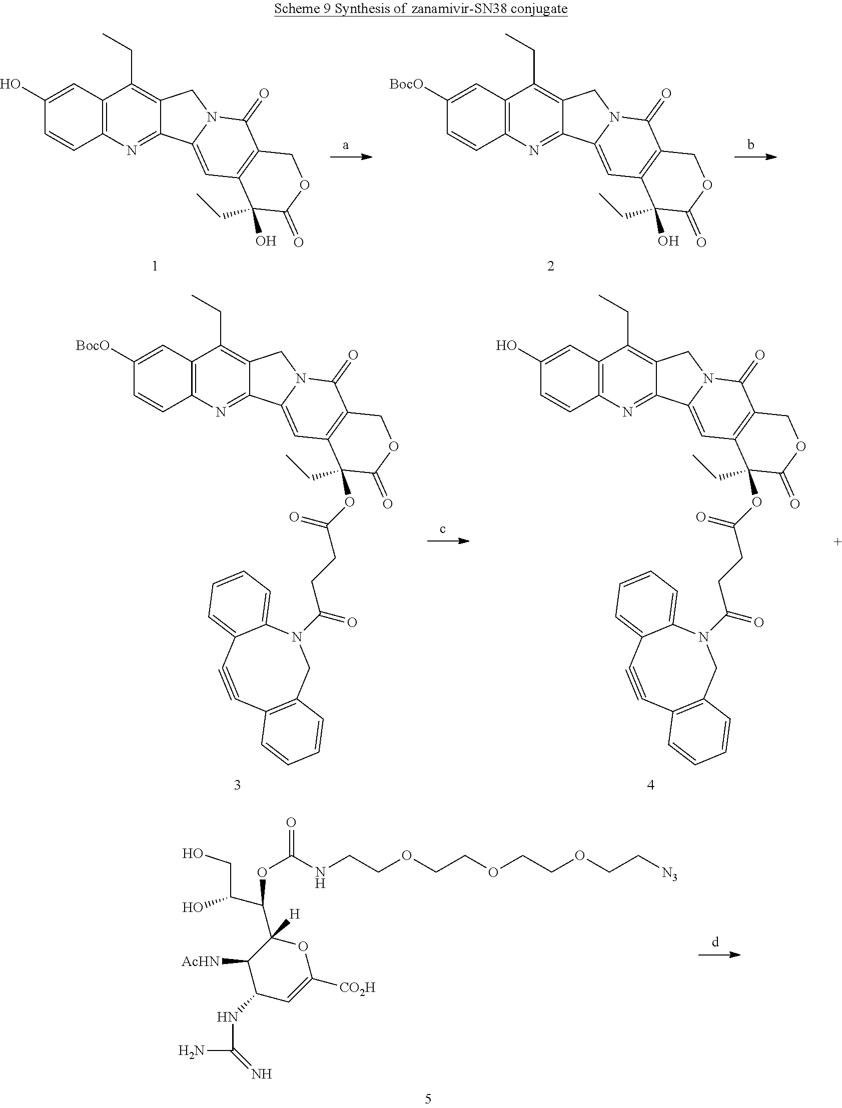

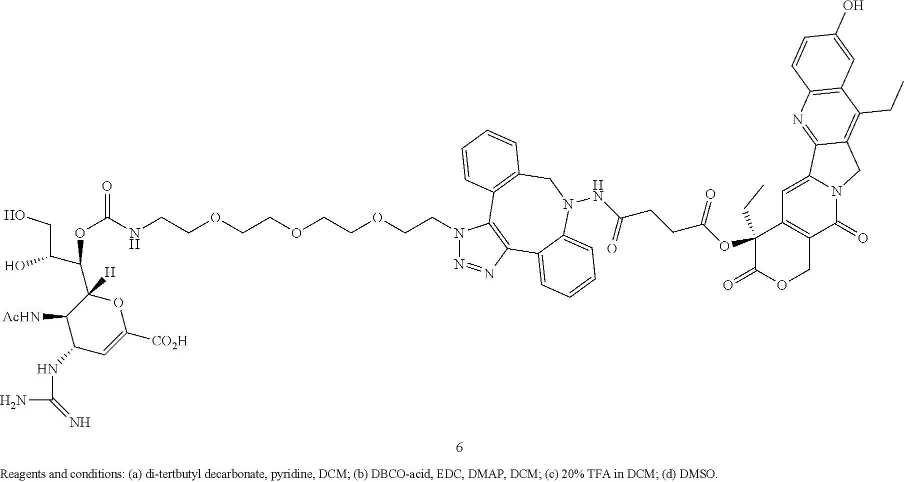 [SMILES: CCc1c2c(nc3ccc(O)cc13)-c1cc3c(c(=O)n1C2)COC(=O)[C@@]3(CC)OC(=O)CCC(=O)NN1Cc2ccccc2-c2c(nnn2CCOCCOCCOCCNC(=O)O[C@@H]([C@@H]2OC(C(=O)O)=C[C@H](NC(=N)N)[C@H]2NC(C)=O)[C@H](O)CO)-c2ccccc21]

### Example 3. Small Molecule Ligand-Targeted Drug Conjugates for Anti-Influenza Chemotherapy

### In Vitro Binding to Influenza Virus Infected MDCK Cells

(1) Confocal Microscope Study with Zanamivir-Rhodamine Conjugate

Method: MDCK cells were seeded in confocal plates and incubated overnight. In the next day when the cells reached 80% confluence, they were infected with 100 TCID₅₀ influenza virus A/Puerto Rico/8/34 (H1N1). On the third day, the infected MDCK cells were incubated with 50 nM zanamivir-rhodamine conjugate in the presence or absence of 5 μM zanamivr. After incubated for 1 h at 37° C., the cells were washed with the cell culture medium and sent to the confocal microscope.

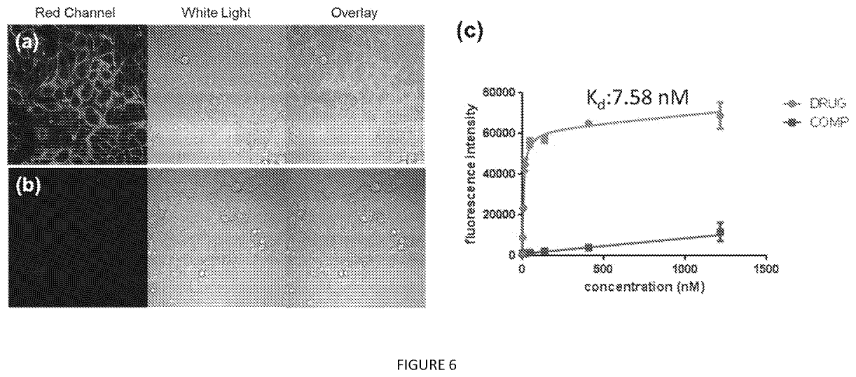

As shown in FIG. 6a, a strong fluorescent signal is observed when influenza virus infected MDCK cells were incubated with 50 nM zanamivir-rhodamine conjugate. The fluorescent emission signal disappears when the binding of zanamivir-rhodamine conjugate to neuraminidase is competed by 100 fold excess of zanamivir (FIG. 6b), which indicates that the cell uptake of the conjugate was receptor mediated. In short, this result demonstrates that zanamivir-rhodamine conjugate can bind to and be internalized into the influenza virus infected MDCK cells.

(2) Binding Affinity Study with Zanamivir-Rhodamine Conjugate

Method: MDCK cells were seeded in 24-well plates and incubated overnight. In the next day when the cells reached 80% confluence, they were infected with 100 TCID₅₀ influenza virus A/Puerto Rico/8/34 (H1N1). On the third day, the infected MDCK cells were incubated with various concentrations of zanamivir-rhodamine conjugate in the presence or absence of 100-fold excess of zanamivr. After incubated for 1 h at 37° C., the cells were washed with the cell culture medium and the remaining fluorescence was quantitated by fluorescence spectroscopy. Apparent K_{d} was calculated by plotting cell bound fluorescence intensity versus the concentration of zanamivir-rhodamine conjugate added using GraphPad Prism 4.

As shown in FIG. 6c, the binding of zanamivir-rhodamine conjugate to neuraminidase expressed on virus infected cells was found to be saturated with K_{d} of 10.98 nM, and this binding of zanamivir-rhodamine conjugate can be competed by 100 fold excess of zanamivir.

Based on the confocal and binding affinity studies above, the zanamivir derivative is proved to be a good candidate as a targeting ligand for the influenza virus neuraminidase.

### Example 4. In Vivo Biodistribution

(1) Bind Affinity Study with ^{99m}Tc Chelated Zanamivir-EC20 Head Conjugate

Method: MDCK cells were seeded in 24-well plates and incubated overnight. In the next day when the cells reached 80% confluence, they were infected with 100 TCID₅₀ influenza virus A/Puerto Rico/8/34 (H1N1). On the third day, the infected MDCK cells were incubated with various concentrations of 99mTc chelated zanamivir-EC20 head conjugate in the presence or absence of 100-fold excess of zanamivr. After incubated for 1 h at 37° C., the cells were washed with the cell culture medium and the radioactivity of the remaining ^{99m}Tc chelated zanamivir-EC20 head conjugate was quantitated by gamma counter. Apparent K_{d} was calculated by plotting cell bound radioactivity versus the concentration of radiotracer using GraphPad Prism 4.

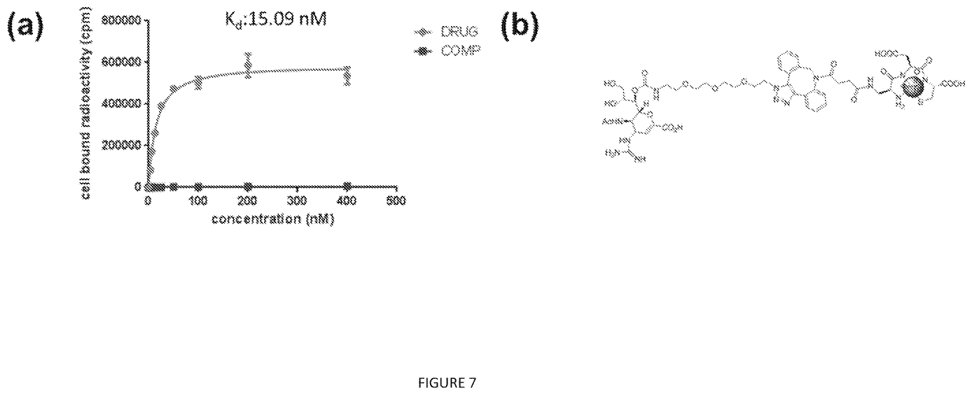

The binding of technetium-99m (^{99m}Tc) chelated zanamivir-EC20 head conjugate to neuraminidase expressed on virus infected cells was found to be saturated with K_{d} of 15.09 nM, and this binding of ^{99m}Tc chelated zanamivir-EC20 head conjugate can be competed by 100 fold excess of zanamivir (FIG. 7a). This binding affinity value is consistent with the value measured by zanamivir-rhodamine conjugate, which further proves that the zanamivir derivative is a good targeting ligand for influenza virus neuraminidase.

(2) Biodistribution Study with ^{99m}Tc Chelated Zanamivir-EC20 Head Conjugate

Method: BALB/c mice (6-7 weeks old) were first intranasally infected with 50 μL influenza virus A/Puerto Rico/8/1934 (H1N1) to develop influenza symptom. 3 days later, the mice were intravenously injected with 100 μL 10 nmol zanamivir-EC20 head conjugate (contains 20 pM ^{99m}Tc chelated conjugate) in the presence or absence of 100-fold excess of zanamivir. 5 h post-injection, major tissues/organs were removed and the amount of radioactivity was determined by gamma counter.

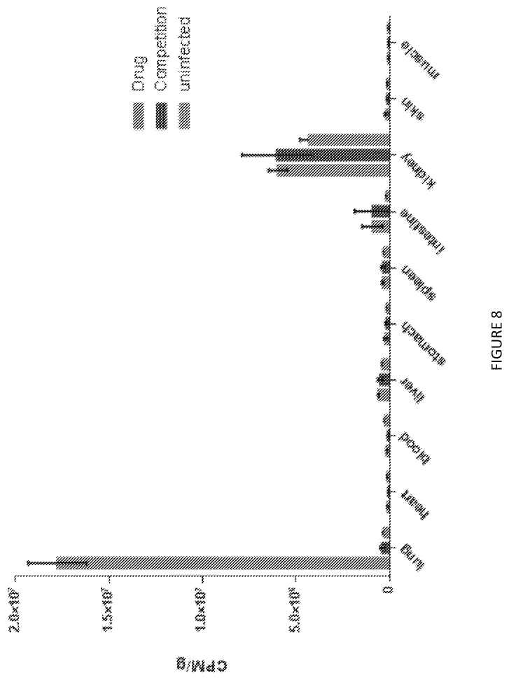

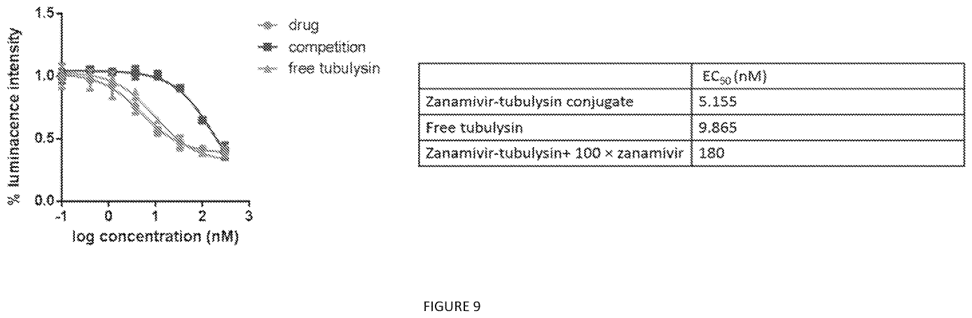

In order to test the ability of zanamivir derivative to specially deliver therapeutic or imaging agents to influenza virus infected lung, the biodistribution profiles of ^{99m}Tc chelated zanamivir-EC20 head conjugate in virus infected mice/uninfected mice were measured. As shown in FIG. 8, the conjugate exhibits the highest uptake in the lung of virus infected mice, the major organ in which influenza virus proliferate. Moreover, there is no lung uptake of ^{99m}Tc chelated zanamivr-EC20 conjugate from the mice in either competition group or uninfected group, indicating that the lung uptake of the conjugate was receptor mediated. Apart from the virus infected lung, kidney is the only organ that shows significant radioactive signal. However, the signals were not abolished in competition group and uninfected group, indicating that the accumulation of ^{99m}Tc chelated zanamivir-EC20 head conjugate in kidney was not neuraminidase mediated. Taken together, this result provides the strong evidence that the zanamivir derivative designed in this project can be used as the targeting ligand to specifically deliver therapeutic or imaging agents to the virus infected lung.

### Example 5. In-Vitro Cytotoxic Study with Zanamivir-Tubulysin B Hydrazide Conjugate

Method: Neuraminidase transfected HEK 293 cells were seed at 96 well plates and incubated with zanamivir-tubulysin B hydrazide conjugate, free tubulysin B hydrazide or zanamivir-tubulysin B hydrazide conjugate in the presence of 100-fold excess of zanamivir for 2 h at 37° C. Cells were then washed with fresh medium and incubated for another 48 h at 37° C. The cell viability was measured using ATP detection (CellTiter Glo, Promege Inc. Madison, WT). EC₅₀ values were calculated by plotting % luminescence intensity versus log concentration of drugs using GraphPad Prism 4.

To determine the cytotoxicity and targeting specificity of zanamivir-tubulysin B hydrazide conjugate, an in-vitro cytotoxic assay using neuraminidase transfected HEK293 cells was performed. As shown in FIG. 9, The EC₅₀ of zanamivir-tubulysin B hydrazide conjugate was 5.2 nM, which is comparable to that of free tubulysin B hydrazide (9.9 nM). Blocking of neuraminidase binding sites with 100-fold excess of zanamivir reduced the cytotoxicity >30-fold, suggesting that most of the cell killing is receptor mediated.

### Example 6. Small Molecule Ligand-Targeted Drug Conjugates for Anti-Influenza Chemotherapy and Immunotherapy

### Competitive Binding of Zanamivir-DNP Conjugate to Neuraminidase Transfected HEK293 Cells Using Zanamivir-Rhodamine Conjugate as the Labelled Ligand

Method: Neuraminidase transfected HEK 293 cells were seed at 24 well plates and incubated overnight. In the next day the cells were incubated with a single concentration of labeled ligand (15 nM zanamivir-rhodamine conjugate) as well as with various concentrations of zanamivir-DNP conjugate or zanamivir. After incubated for 1b, the cells were washed with the cell culture medium and the remaining fluorescence was quantitated by fluorescence spectroscopy. Apparent K_{d} was calculated by plotting cell bound fluorescence intensity versus the log concentration of zanamivir-DNP conjugate or zanamivir added using the competition binding equation in GraphPad Prism 4.

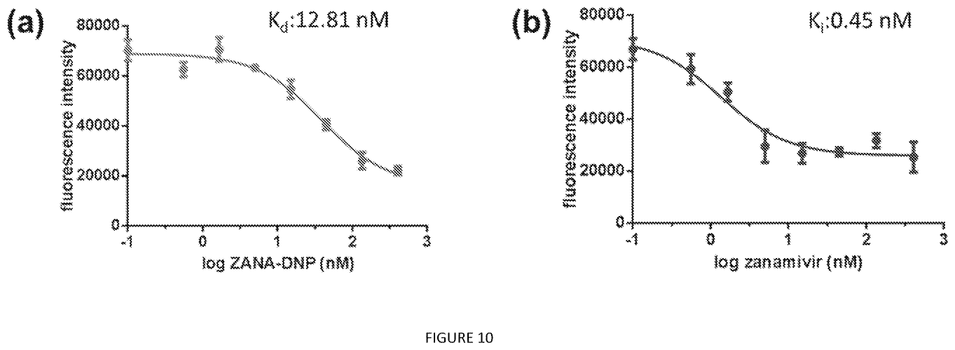

The binding affinity of zanamivir-DNP conjugate to cell membrane bound neuraminidase was measured in a competitive binding experiment. FIG. 10 shows that the binding affinity of zanamivir-DNP conjugate and zanamivir were measured at 12.81 and 0.45 nM, respective. Even though the binding affinity of the targeting ligand drops 28-fold upon being attached with DNP moiety, its binding affinity is still in a low nanomolar range, which indicates that zanamivir-DNP conjugate has the potential to be used as the ligand targeted hepten conjugate.

### Example 7. Anti-DNP Antibody Recruiting Assay with Zanamivir-DNP Conjugate

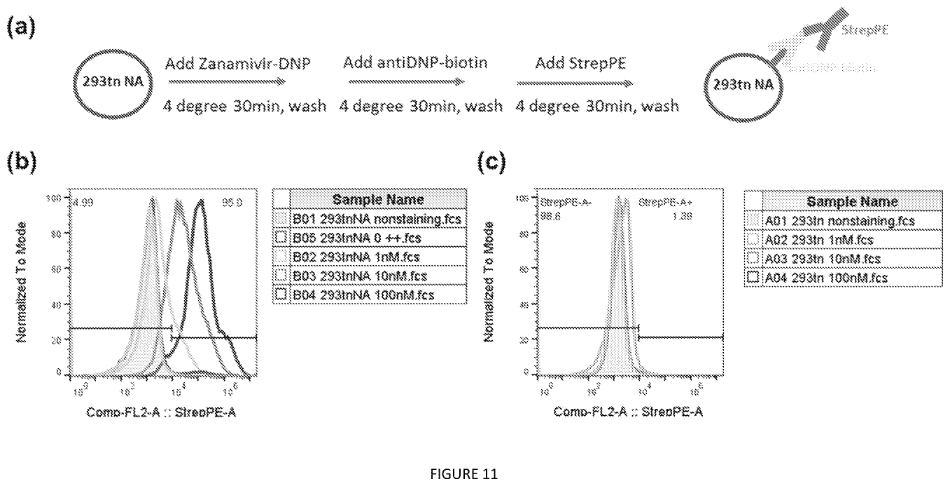

As shown in FIG. 11, zanamivir-DNP conjugate was able to specifically bind to neuraminidase on the neuraminidase transfected HEK293 cell membrane (293tn NA) and recruit antiDNP antibody at around 10 nM. No binding was observed in non-transfected HEK293 cells (293tn).

Methods: 293tn NA (Neuraminidase transfected) and control (non-transfected 293tn) cells were incubated with different concentrations of zanamivir-DNP conjugate at 4° C. for 30 min, followed by PBS washing for 3 times. After that, cells were stained with antiDNP-biotin and Streptavidin-PE at 4° C. for 30 min. Cells were washed by PBS for 3 times and submitted to flow cytometry.

### Example 8. Mouse Protection Study

Method: BALB/c mice (4 weeks old) were immunized with DNP-KLH on week 1 and week 3 twice. On week 4, both the immunized and unimmunized mice were intranasally infected with 50 μL 100 LD₅₀ influenza virus A/Puerto Rico/8/1934 (H1N1) to develop the influenza symptom. 2 h later, mice were intranasally given PBS/zanamivir/zanamivir-DNP conjugate (1.5 μmol/kg) one time a day for five days. Mice were counted as dead when losing either 25% of their initial weight or when they were moribund.

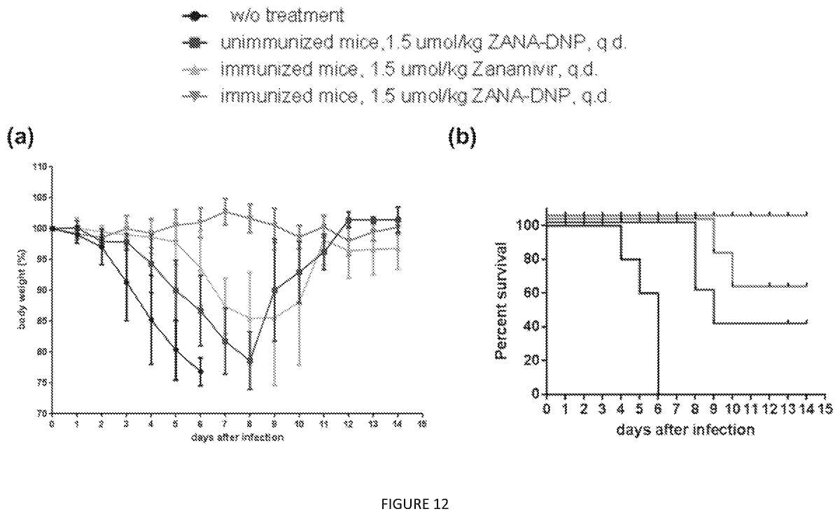

As shown in FIG. 12, zanamivir-DNP conjugate (red invert triangles) has a superior effect to zanamivir (green triangles) at dose of 1.5 μmol/kg. There was no body weight loss or influenza symptom observed for zanamivir-DNP conjugate treated immunized mice (FIG. 12a, red line). Zanamivir-DNP conjugate protected all immunized mice from lethal virus challenge (FIG. 12b, red line). In contrast, zanamivir only rescued 60% of the immunized mice (FIG. 12b, green line). It is worth mentioning that the efficacy of zanamivir-DNP conjugate dropped dramatically when it was given to the unimmunized mice (blue line), which underlines the importance of the immunological function of zanamivir-DNP conjugate.

### Example 9. Influenza Virus Induced Tumor Types for Targeted CAR T Therapy

In this example, in vitro study of CAR T cell killing NA expressing HEK 293 cells is demonstrated.

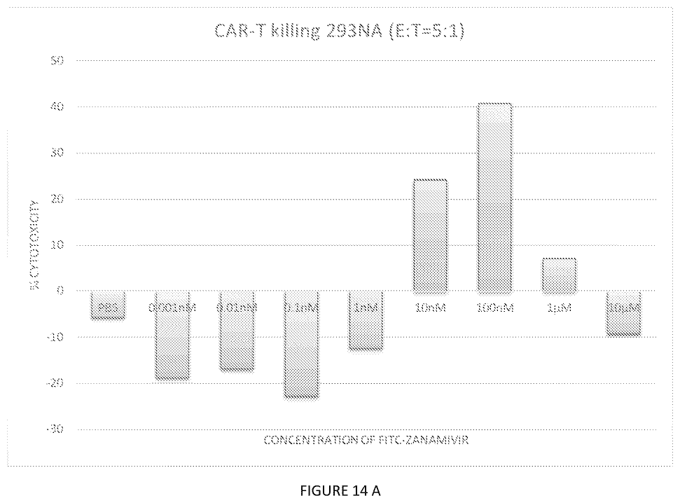

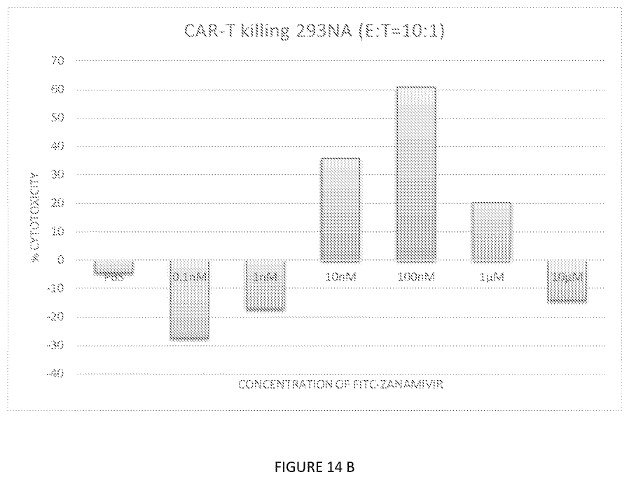

FIG. 14 shows that HEK 293 cells that express NA (293NA) are used to mimic influenza virus infected cells. FIGS. 14A and B respectively show
CAR-T (100,000 cells): 293NA (20,000)=5:1, causes 41% killing
CAR-T (200,000 cells): 293NA (20,000)=10:1, causes 61% killing

### Example 10. No Binding of Zanamivir-FITC to Normal 293T Cells

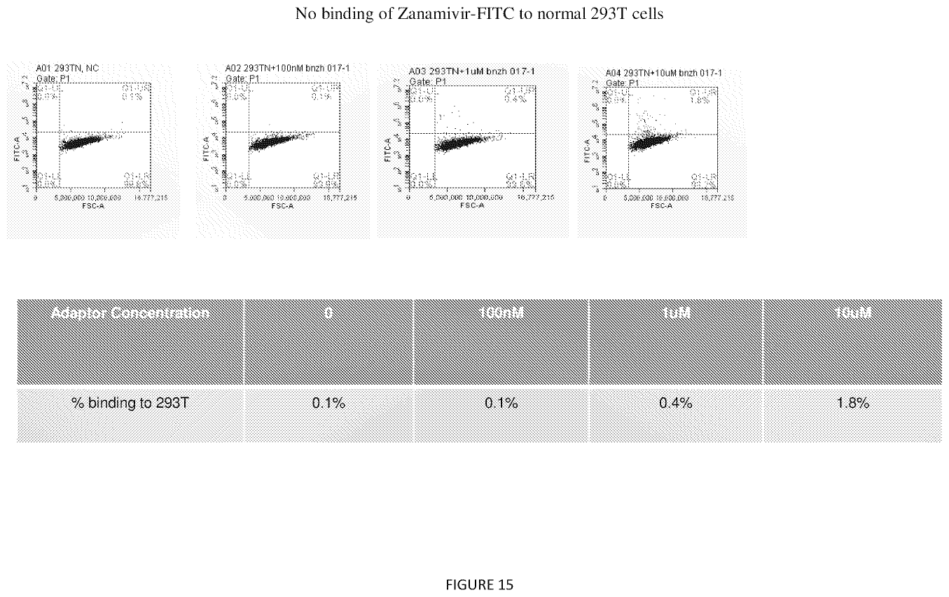

FIG. 15 shows zanamivir-FITC does not bind to normal 293 T cells. Because of the specific binding between zanamivir and NA on a cell surface, if 293 cells are not transfected with NA expression, zanamivir-FITC will not be on the cell surface, thus normal 293 cells are not detected when gated in flow cytometer. Only cells expressing NA on its surface will be detected by zanamivir-FITC conjugate. Therefore, Zanamivir-FITC may serve as a probe to identify NA expressing cells.

### Example 11. The Cytotoxicity Against NA is Specifically Induced by Zanamivir-FITC

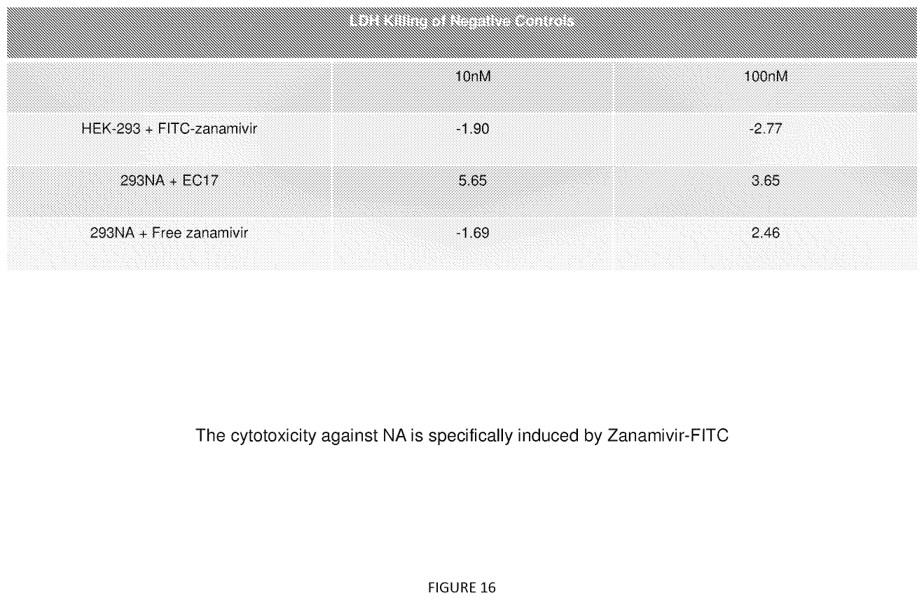

FIG. 16 shows anti-FITC CAR T executes its cytotoxicity against HEK 293NA cells specifically.

three different groups (HEK-293+FITC-zanamivir, 293NA+EC17, 293NA+Free zanamivir) were co-cultured with human CAR-T cells and the % of killing were tested by LDH.

In this table/FIG. 16, HEK-293 are normal 293T cells that do not express NA, while 293NA are 293T cells that express NA. FITC-zanamivir is the adaptor designed for the CAR-T to target cells that express NA (in nature, influenza infected cells; in this experiment, 293NA). For EC17, the FITC side can bind to CAR-T cells, but the other side do not bind to 293NA. Therefore, the three groups can be considered as: 1. non-target cells with correct adaptor; 2. target cells with wrong adaptor; 3. target cells with free drug. The results of the three groups were expected to be no killing and the experimental results in FIG. 16 support the hypothesis (The 3-5% killing from 293NA+EC17 group could be variation, and it is not significant compared to the 40-60% killing from the experimental group (293NA+FITC-zanamivir) in FIG. 14.

### Example 12. In Vitro Essay Using Real Virus

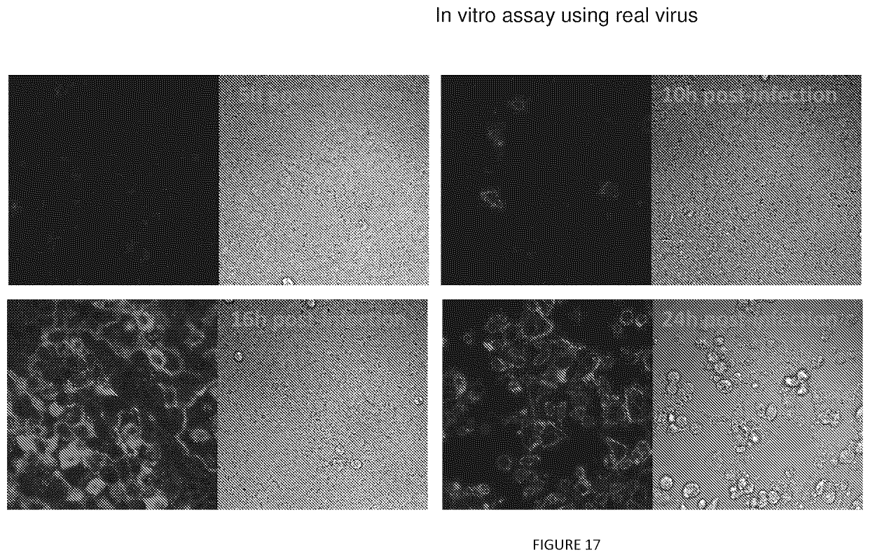

Similar to Example 10, MDCK cells infected by real virus can be identified by zanamivir-rhodamine conjugate to check the expression level of NA, as shown in FIG. 17. Typically, confluent MDCK cells were infected with 100TCID₅₀ influenza virus (H1N1). In exemplified FIG. 17, cells are stained with 100 nM zanamivir-rhodamine conjugate to check the expression level of NA on the cell surface.

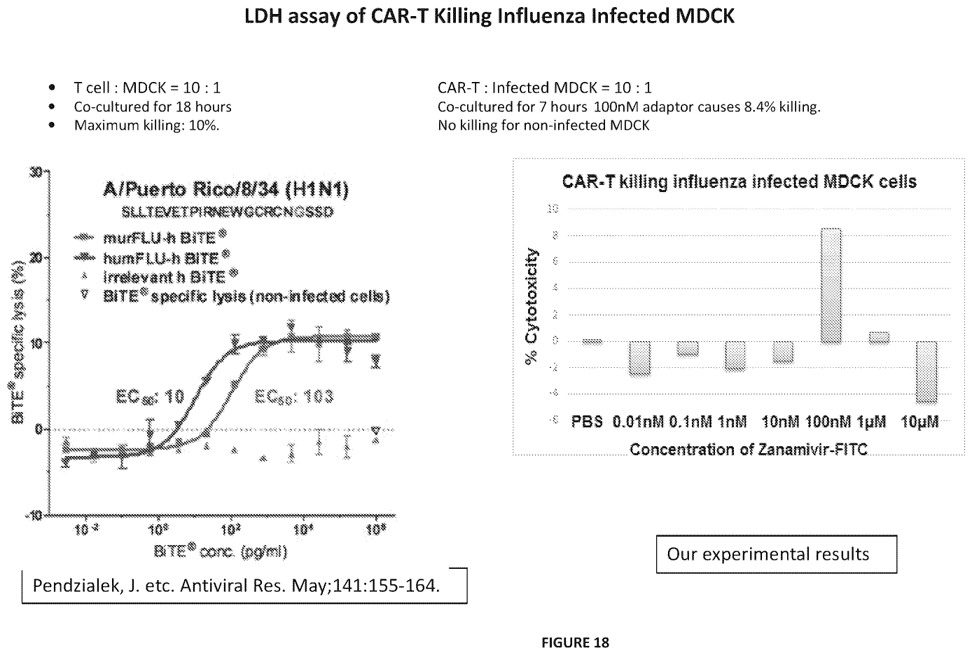

After the influenza virus infection to MDCK cells for about 15 hours, these cells are co-cultured with CAR-T cells for a number of hours. Then the efficacy of the CAR-T killing effect is checked by at least two different ways: one is to check the cell lysis such as LDH assay. FIG. 18 provides zanamivir-FITC conjugate adapted anti-FITC CAR-T cells to kill H1N1 infected MDCK. The left panel shows a literature report of regular T cell killing of MDCK cells at maximum rate of 10% after co-cultured for 18 hours, whereas adapted CAR-T cells caused about 8.4% killing of influenza infected MDCK cells after only 7 hours of co-culture. Further, adapted CAR-T cells do not have non-infected MDCK killing, indicating the CAR T is very specific to infected T cells. The other way of checking CAR T killing effect is to measure the virus titer in the supernatant (for example, using qPCR to measure viral genetic material to quantify viral replication).

### Example 13. In Vivo Mouse Model to Study Influenza Induced Tumor Types for Targeted CAR-T Therapy

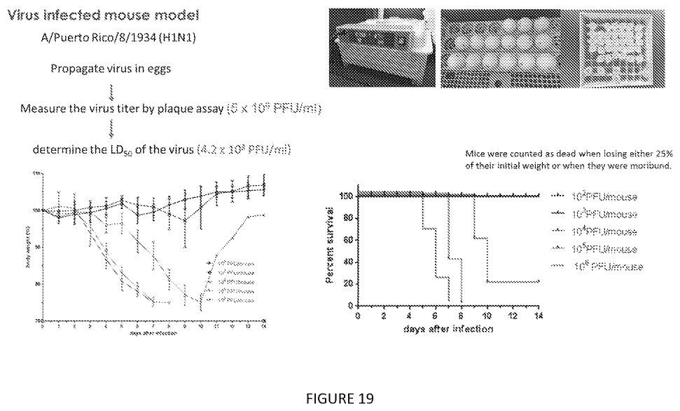

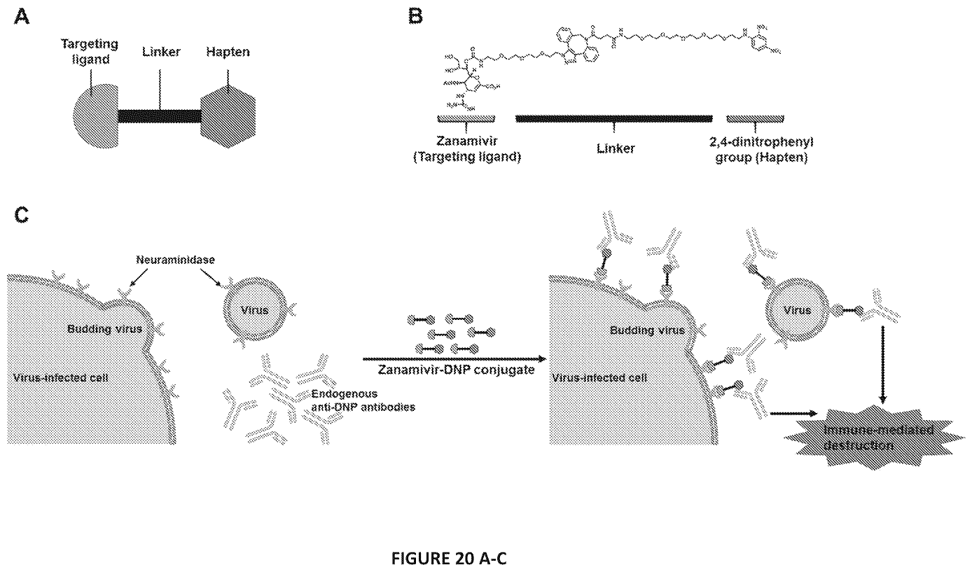

FIG. 19 provides a mouse model for studying influenza virus induced tumor type, using targeted adaptor mediated anti-FITC CAR-T therapy described in previous examples. Influenza virus infected NSG mice are first studied to determine the LD₅₀ of the virus titer. After establishing proper virus titer and infected NSG mice, zanamivir-FITC conjugated adapter and anti-FITC CAR-T cells are applied to the infected NSG mice to rescue. It is expected that mice survival will increase upon the zanamivir-FITC conjugate and anti-FITC CAR-T rescue.

### Example 14. The Binding Affinity of Zanamivir-DNP Conjugate for Both Influenza a Group 1 Neuraminidase (Represented by N1) and Group 2 Neuraminidase (Represented by N2)

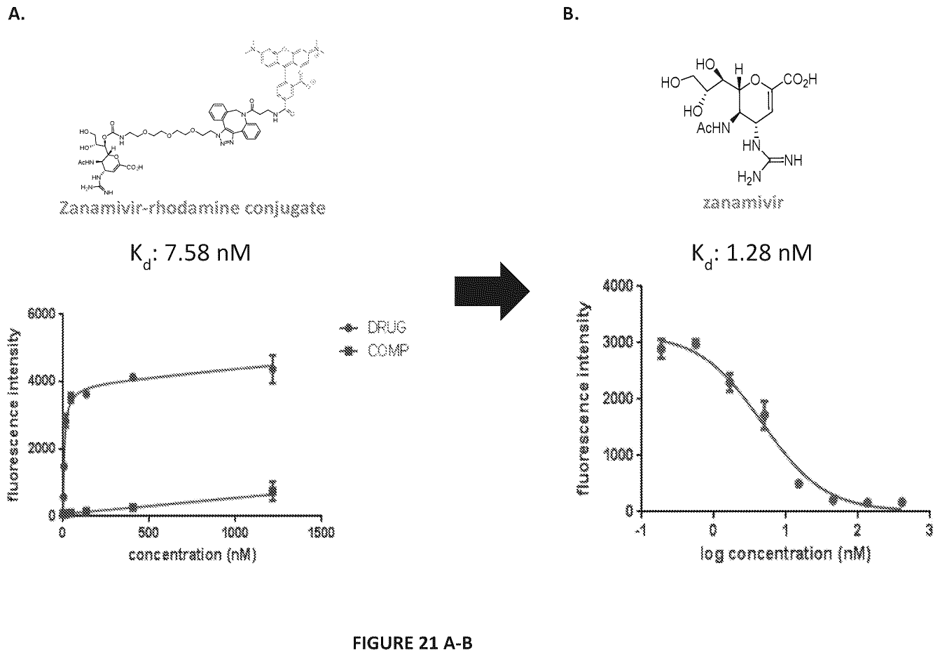

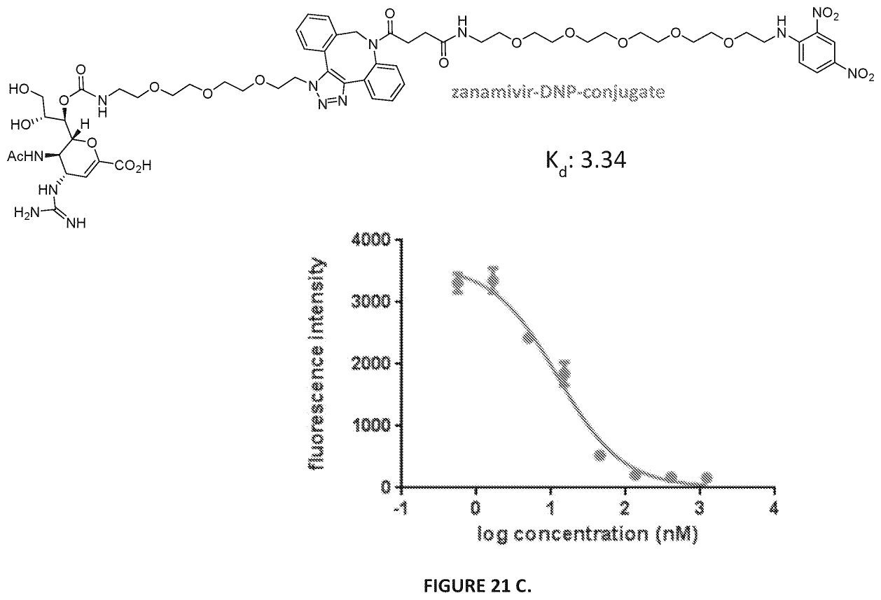

FIG. 21 provides in-vitro binding assay of influenza virus A/Puerto Rico/8/34 (H1N1) infected MDCK cells.

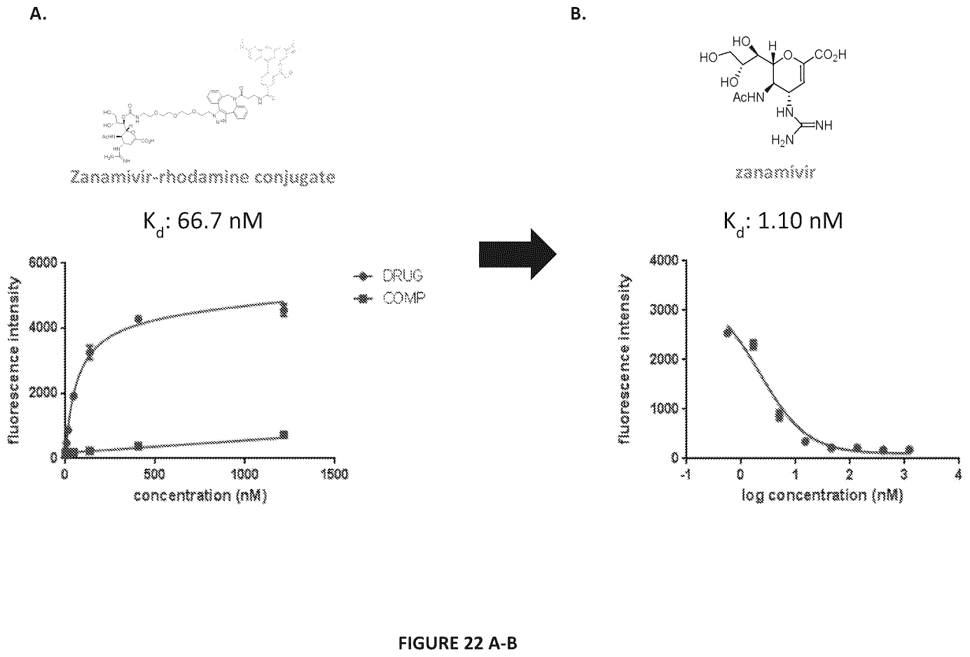

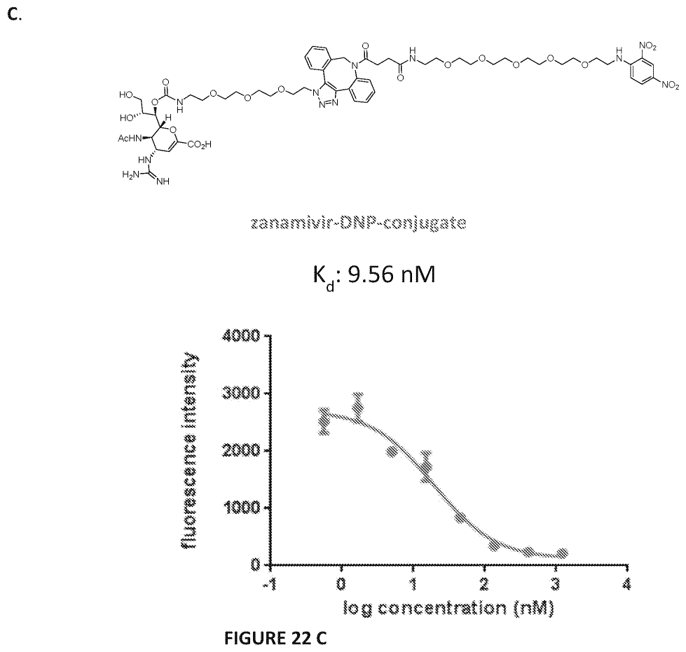

FIG. 22 provides in-vitro binding assay of influenza virus A/Aichi2/1968 (H3N2) infected MDCK cells.

### Example 15. The Ability of the Zanamivir-DNP Conjugate to Induce the Killing of Influenza Virus-Infected Cells Through CDC and ADCP Effects

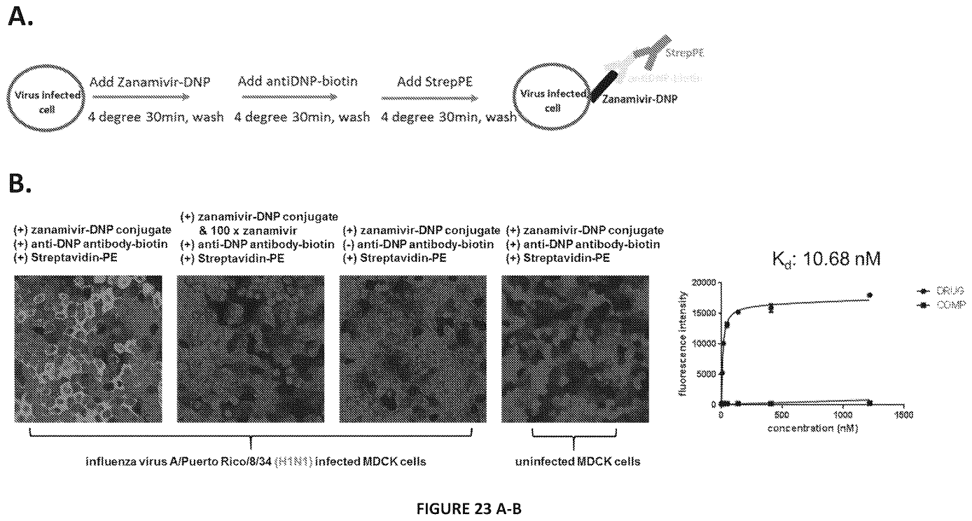

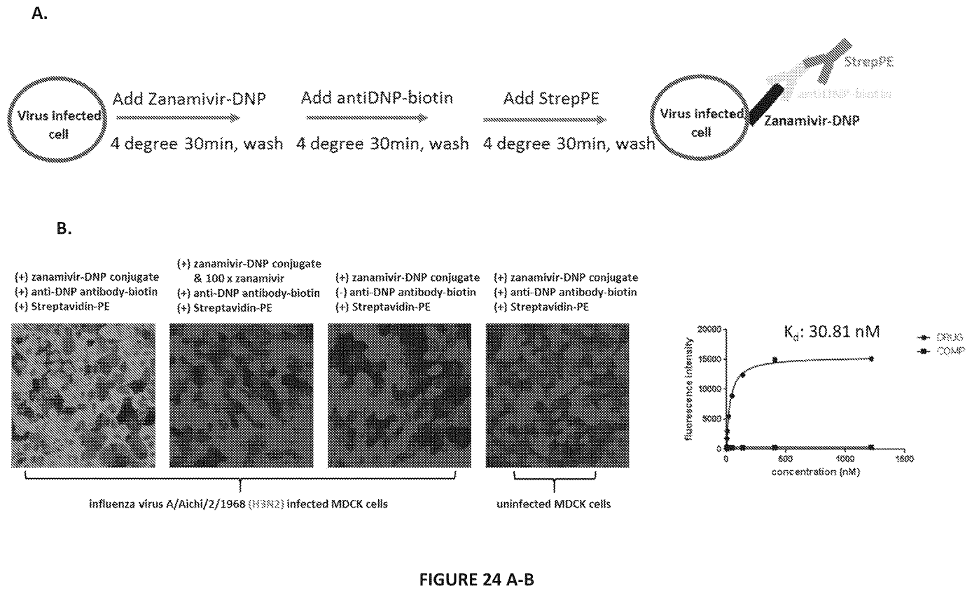

FIGS. 23-24 show that anti-DNP antibody can be recruited to virus infected MDCK cells for both H1N1 and H3N2 strains.

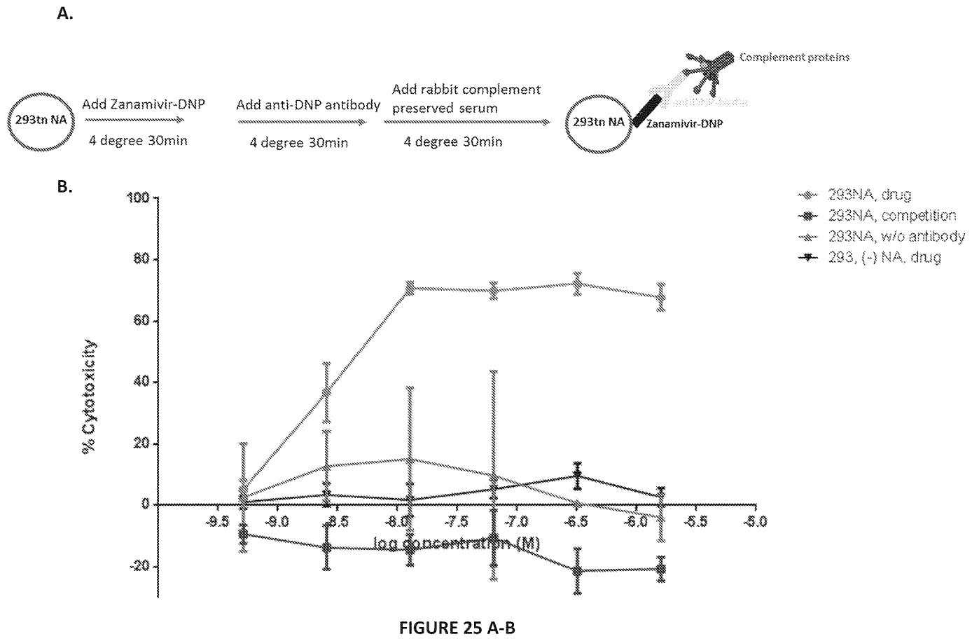

FIG. 25 shows complement dependent cytotoxicity assay of zanamivir-DNP conjugate, in which only 10 nM drug conjugate is needed for achieving maximum killing. This indicates zanamivir-DNP conjugate is much more potent than zanamivir alone.

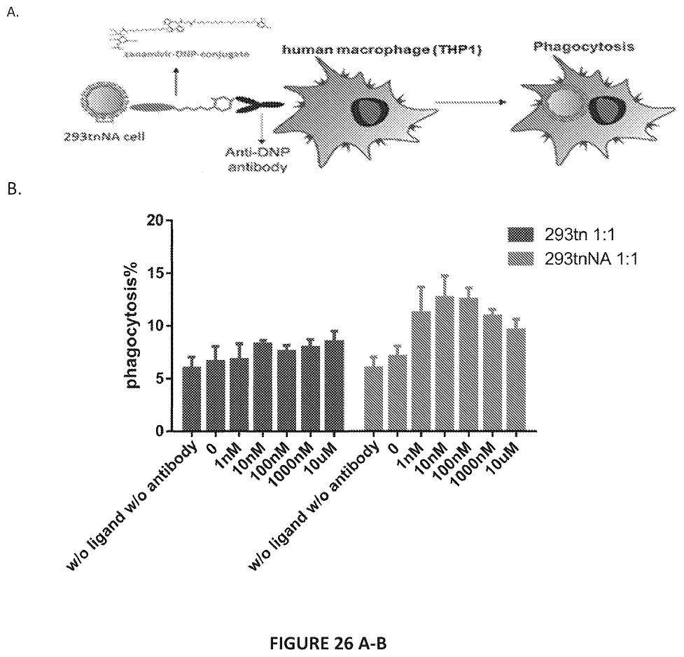

FIG. 26 shows zanamivir-DNP conjugate may bring anti-DNP antibody to achieve antibody dependent phagocytosis (ADCP).

### Example 16. a Series of Live Mouse Studies Demonstrating

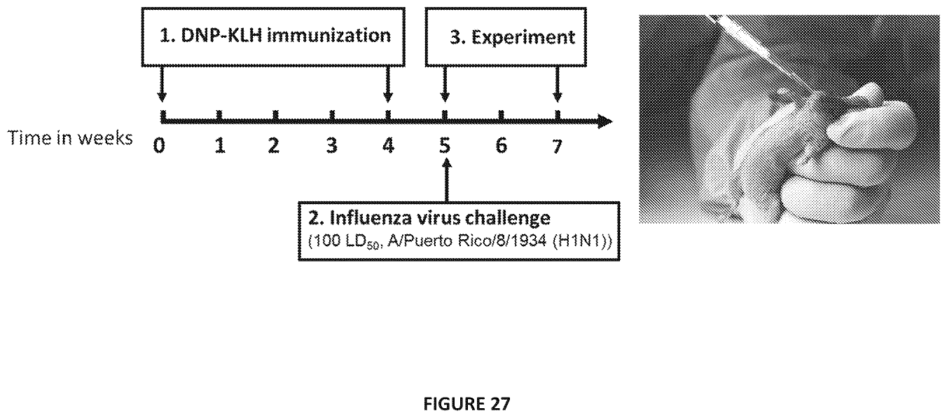

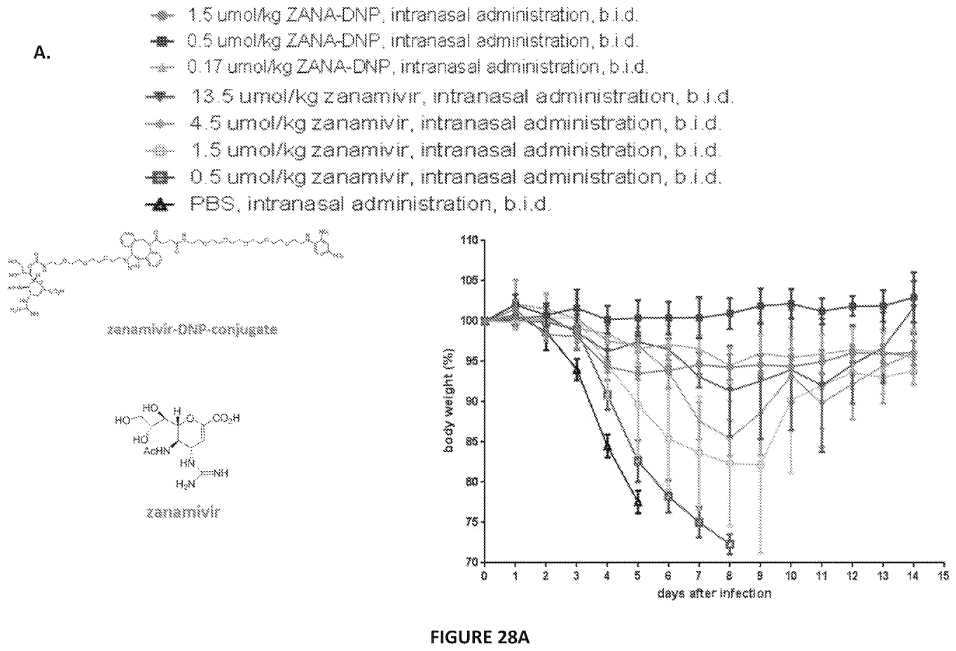

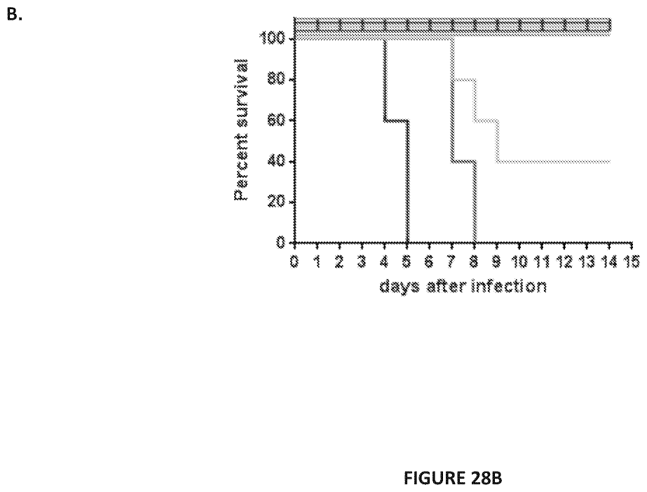

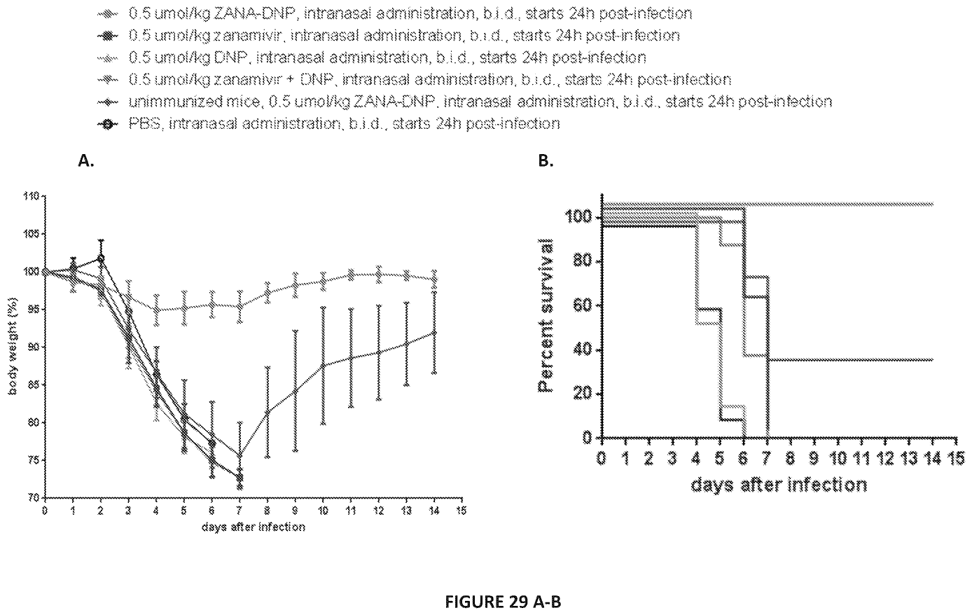

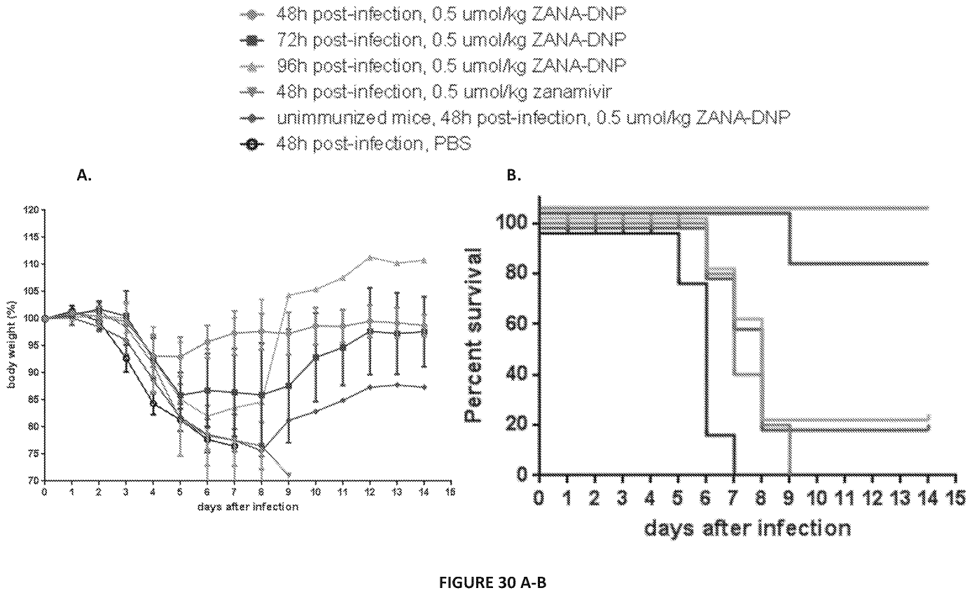

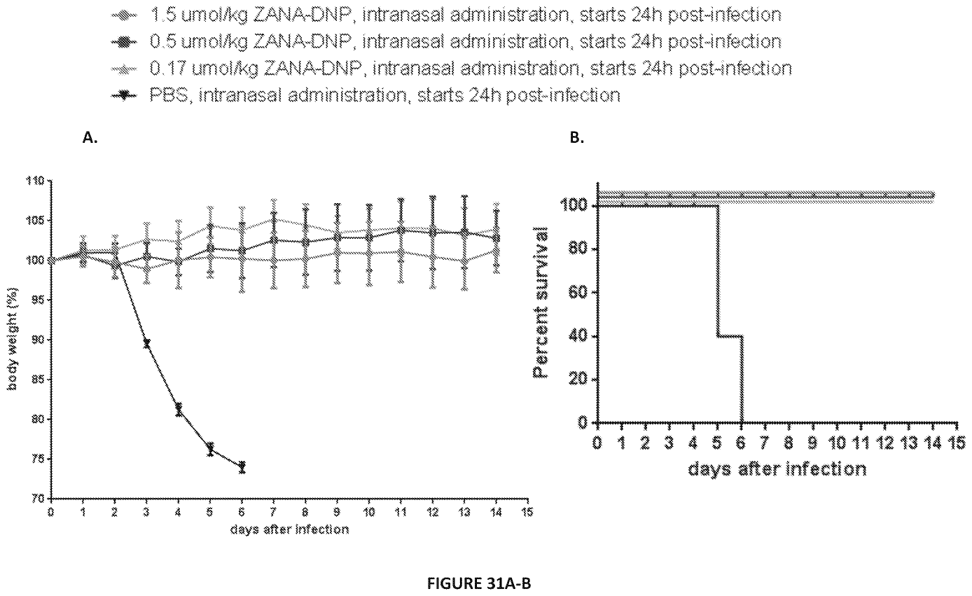

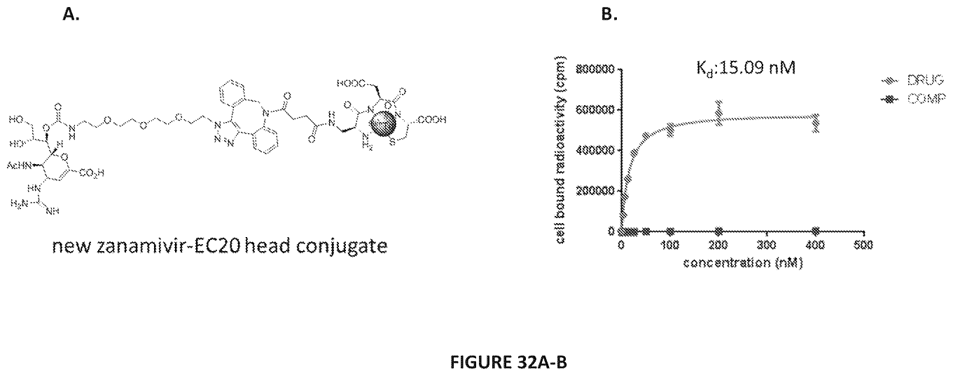

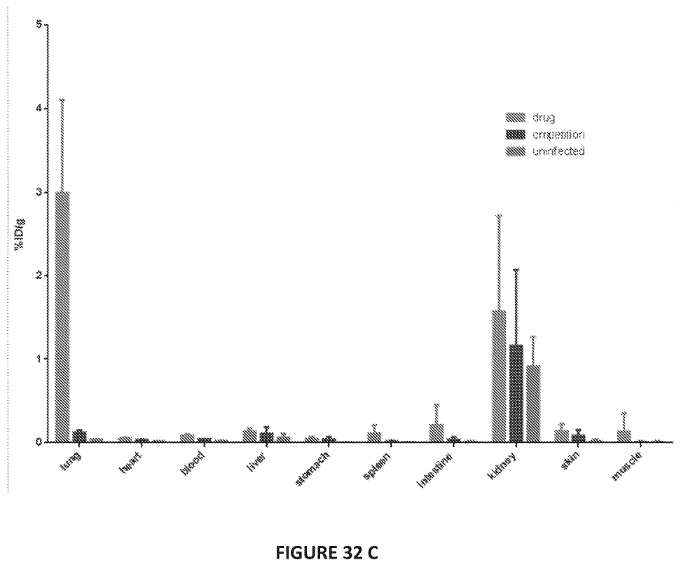

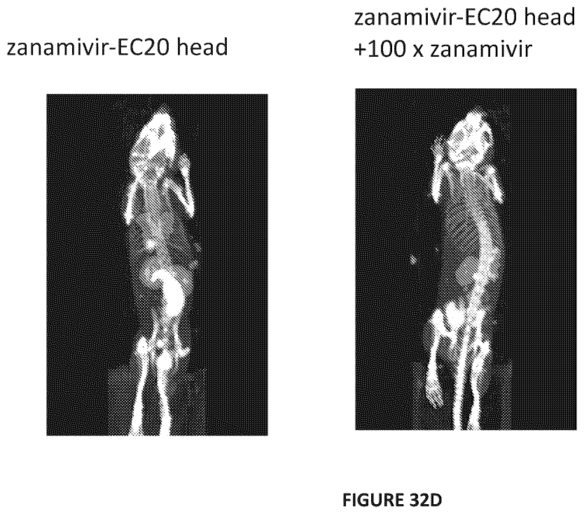

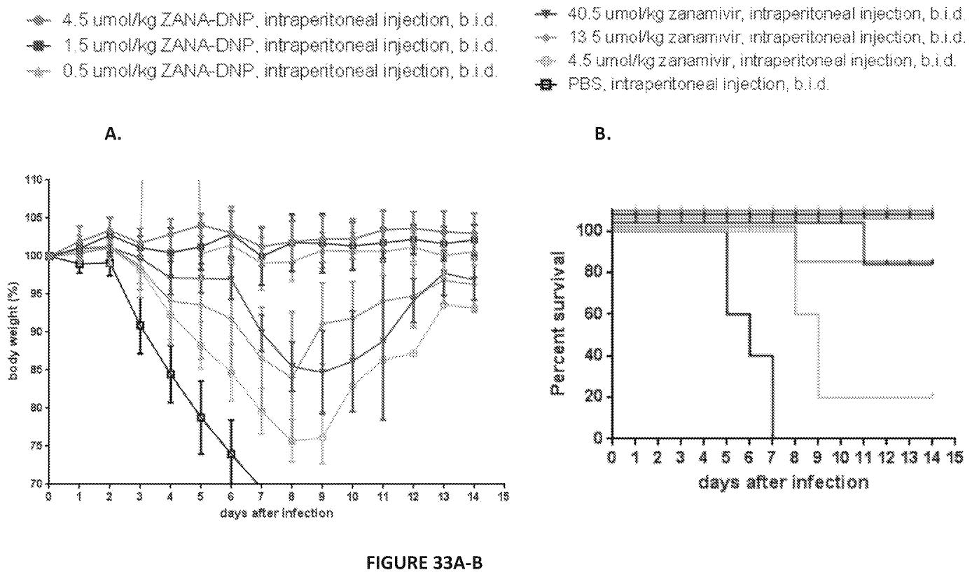

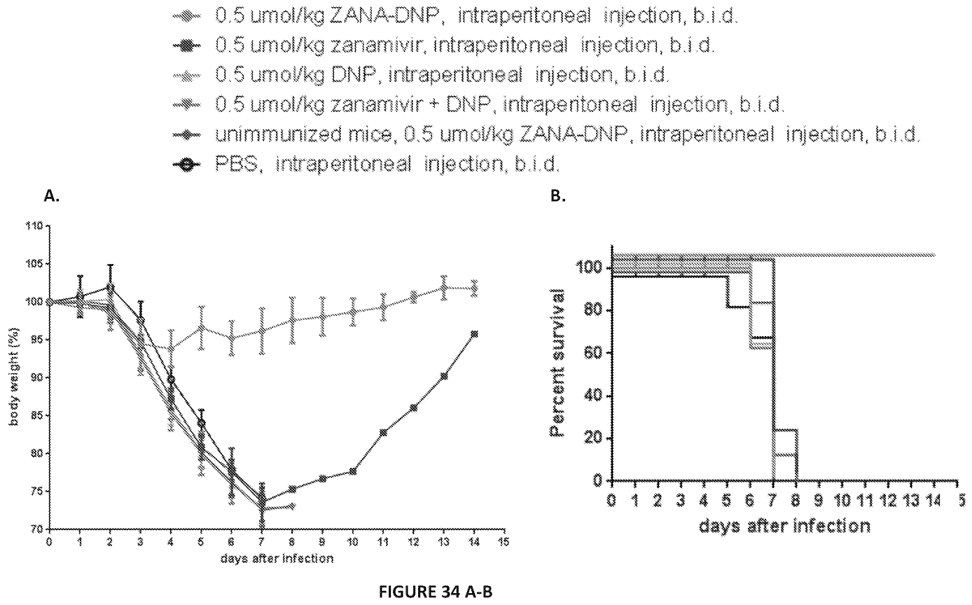

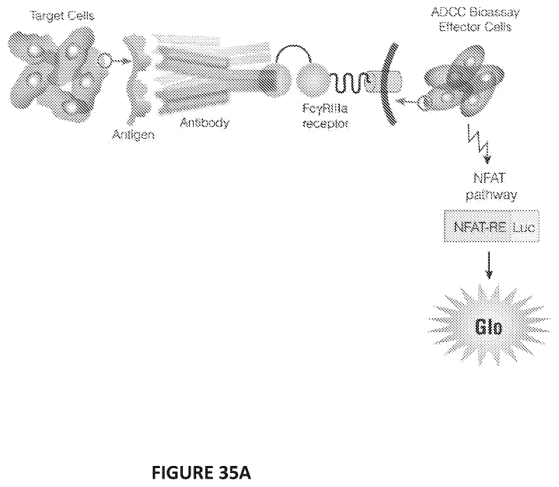

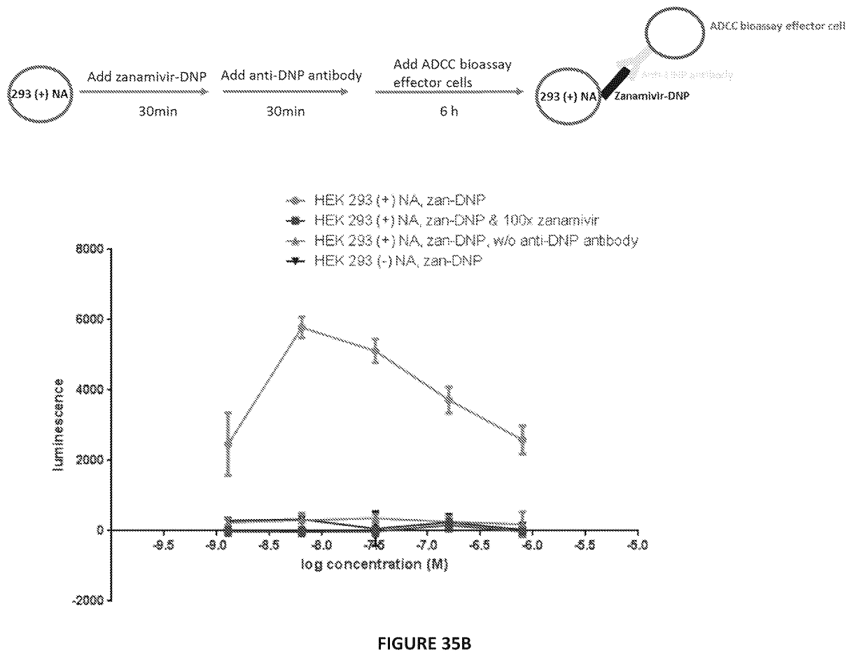

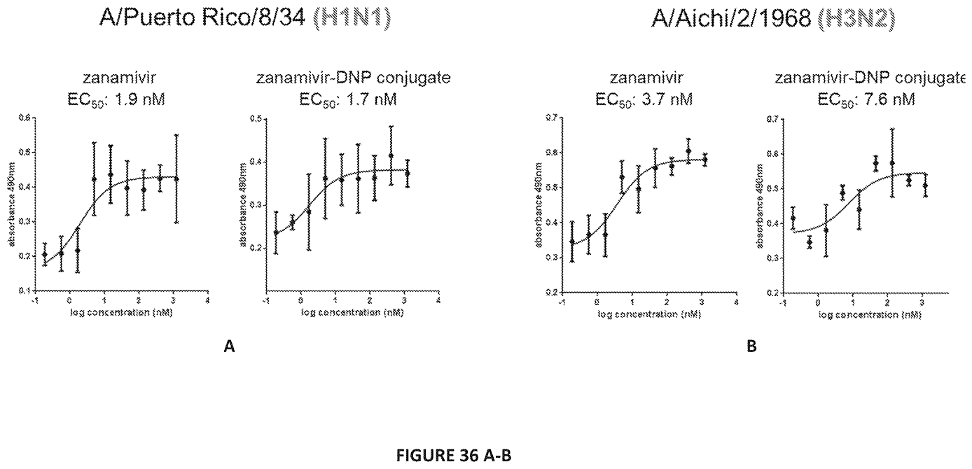

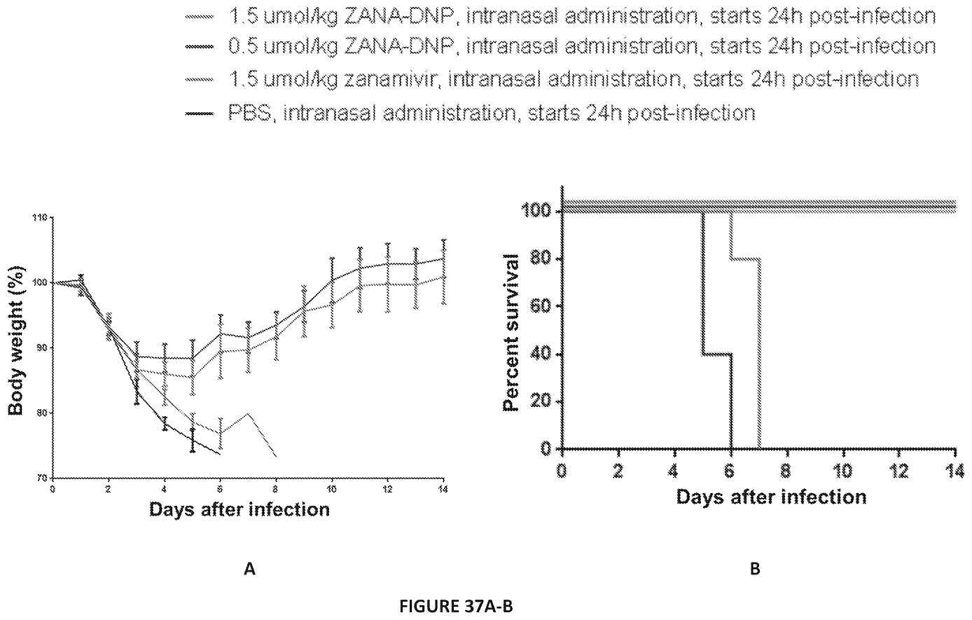

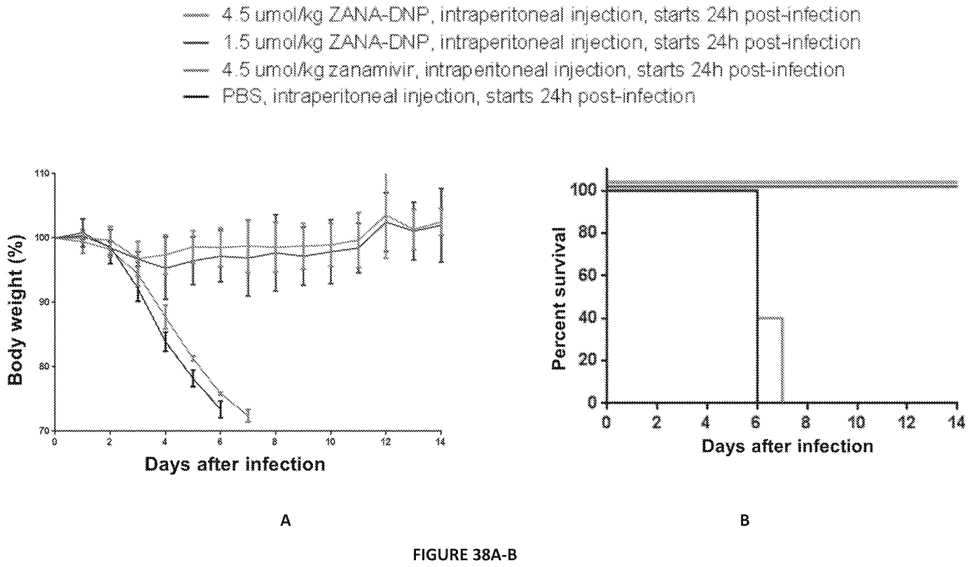

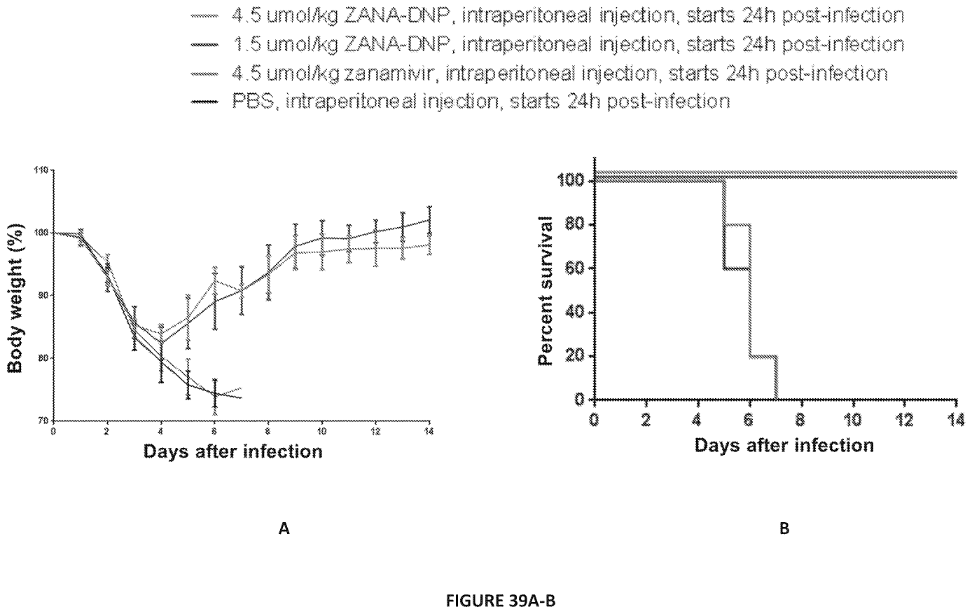

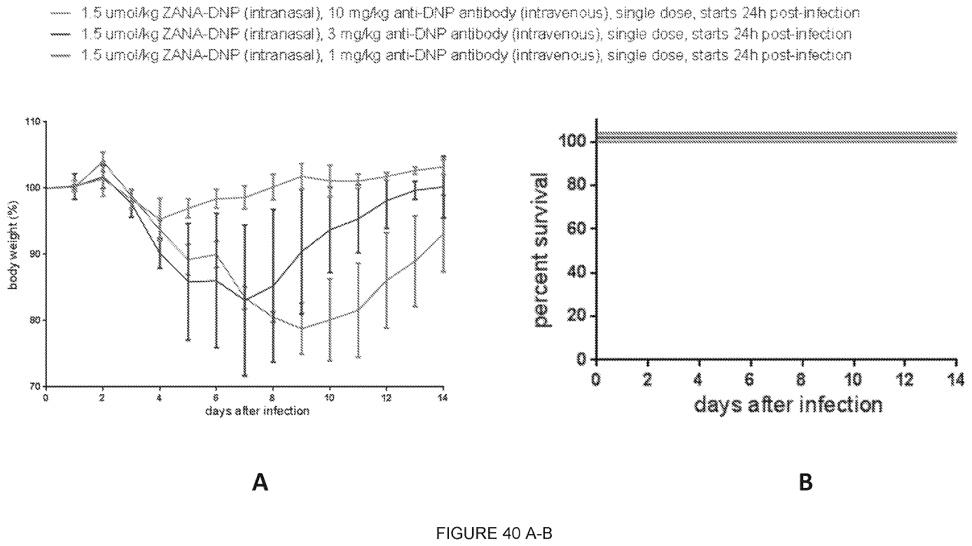

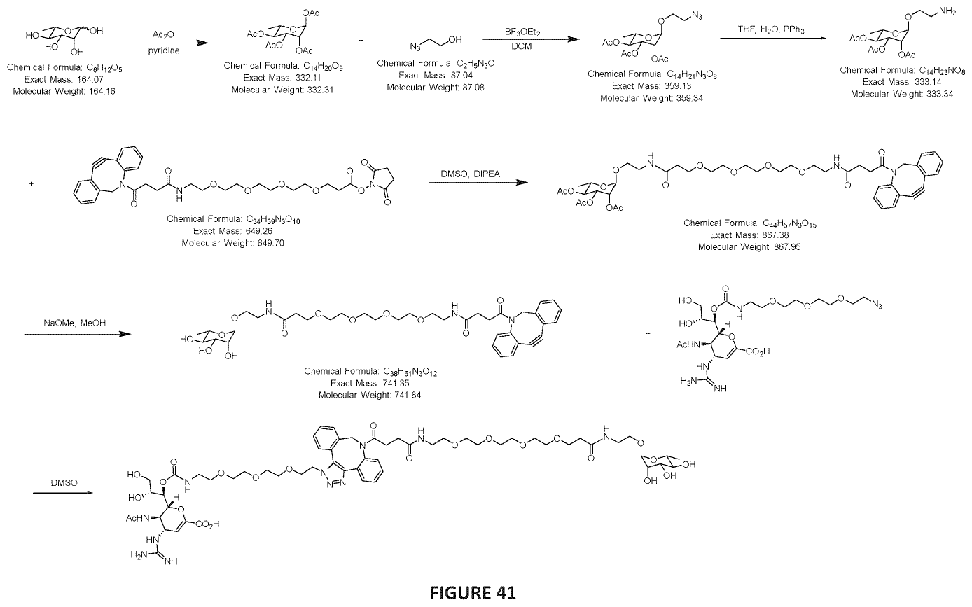

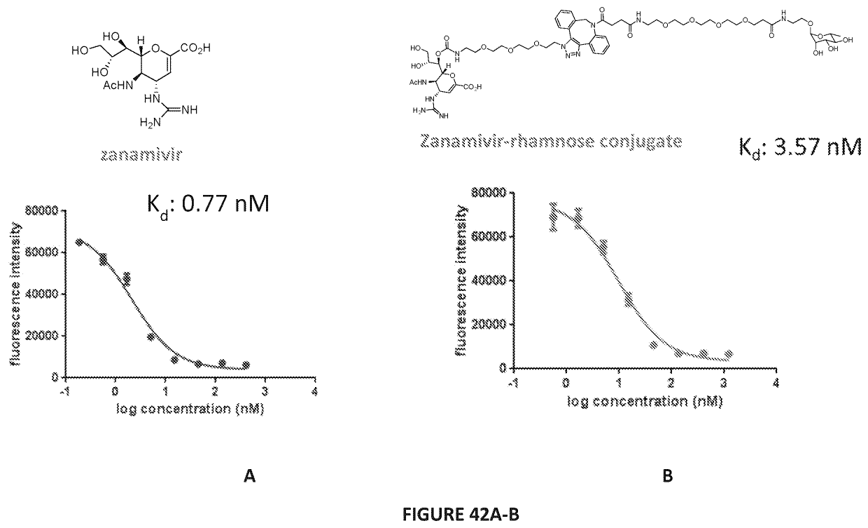

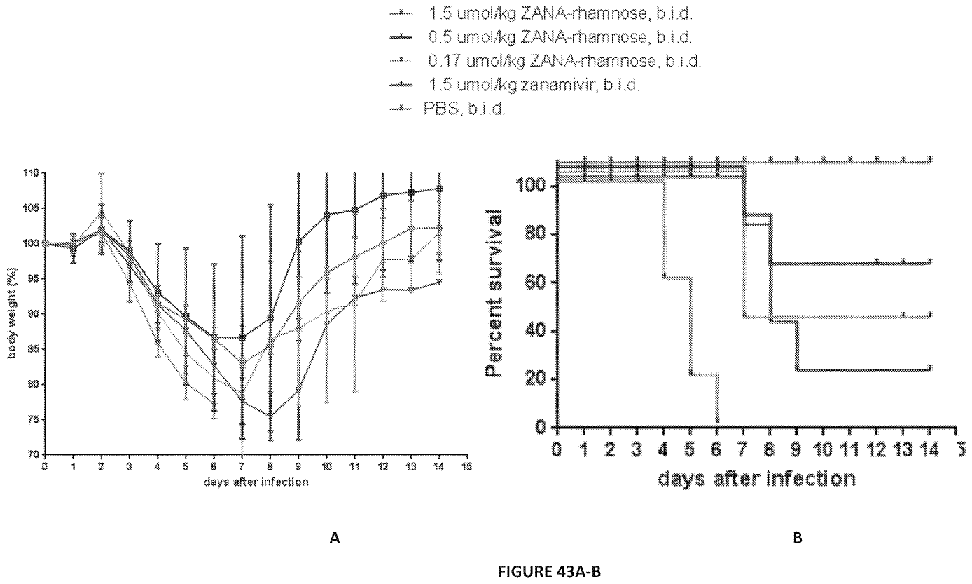

a. Efficacy of intranasal administration of drug in treating influenza virus infection (FIGS. 27-29)
b. Dose escalation study showing optimal dose following intranasal administration (FIG. 27)
c. Dose frequency study showing that a single dose is sufficient to yield complete cures following intranasal administration (FIG. 28, 31)
d. That treatment can be delayed until 72 hours after detection of flu symptoms and complete cures can still be achieved following intranasal administration (FIG. 29-30)
e. The same drug can be administered via intraperitoneal injection and still achieve complete cures (FIGS. 33-34)
f. In all of the above assays, our drug outperforms zanamivir dramatically (FIGS. 27-34)
g. BioD data and spect/CT imaging showing specificity for infected lung tissue (FIG. 32)

### Example 17. Ligand-Targeted Immunotherapy for the Treatment of Influenza Analyzed with Antibody Dependent Cellular Cytotoxicity Reporter Bioassay (FIG. 35A-B)

In this Example, an ADCC reporter bioassay is applied to monitor the zanamivir-DNP conjugate inducted ADCC response via a firefly luciferase reporter assay (ADCC Reporter Bioassays, V Variant, Catalog #: G7010, Promega). Briefly, engineered Jurkat cells are used to stably express FcγRIIIa receptor and an NFAT (nuclear factor of activated T-cells) response element driving expression of firefly luciferase is used as effector cells shown in FIG. 35A. Antibody biological activity in ADCC Mode of Action is quantified through the luciferase produced as a result of NFAT pathway activation, which indicates the engagement of zanamivir-DNP conjugate in mediating ADCC effect to the target cell, as outlined in FIG. 35B. This example shows that zanamivir-DNP conjugate serves important roles for mediating ADCC effects.

### Example 18. Ligand-Targeted Immunotherapy for the Treatment of Influenza Analyzed with In Vitro Antiviral Assay for H1N1 and H3N2 Infected MDCK Cells (FIG. 36A-B)

In this Example, MDCK cells infected with either H1N1 or H3N2 virus were studied for zanamivir-DNP conjugate protection effect. As shown in FIG. 36A (H1N1) and 36B (H3N2), the EC50 represents the inhibitor concentrations for 50% protection of virus infected MDCK.

To assure that zanamivir-DNP conjugate can still inhibit the neuraminidase activity necessary for its suppression of influenza virus proliferation, we compared the potency of zanamivir and zanamivir-DNP conjugate in suppressing propagation of influenza virus in an MDCK-influenza virus co-culture.

### Example 19. Single Dose Treatment (Intranasal Administration) for H3N2 Virus Infected Mice (FIG. 37A-B)

In this Example, DNP-KLH immunized mice (5 mice/group) were infected with 50 μL A/Aichi/2/1968 (HA, NA), x-31b (H3N2) virus (100 LD₅₀) at day 0.

Mice were intranasally given zanamivir-DNP conjugate/zanamivir/PBS 24 h post-infection for only one time and mice were counted as dead when losing either 25% of their initial weight or when they were moribund.

As shown in FIGS. 37A and B, intranasally administered zanamivir-DNP provides good protection 14 days of post infection (all mice were cured), whereas zanamivir alone does not provide the same protection.

### Example 20. Single Dose Treatment (Intraperitoneally Administration) for H1N1 Virus Infected Mice (FIG. 38A-B)

In this Example DNP-KLH immunized mice (5 mice/group) were infected with 50 uL A/Puerto Rico/8/34 (100 LD₅₀, 4.2×10⁵ PFU) at day 0.

Mice were intraperitoneally given zanamivir-DNP conjugate/zanamivir/PBS 24 h post-infection for only one time and mice were counted as dead when losing either 25% of their initial weight or when they were moribund.

As shown in FIGS. 38A and B, intraperitoneally administered zanamivir-DNP provides good protection 14 days of post infection (all mice were cured), whereas zanamivir alone does not provide the same protection.

### Example 21. Single Dose Treatment (Intraperitoneally Administration) for H3N2 Virus Infected Mice (FIG. 39A-B)

In this Example DNP-KLH immunized mice (5 mice/group) were infected with 50 uL A/Aichi/2/1968 (HA, NA), x-31b (H3N2) virus (100 LD₅₀) at day 0.

Mice were intraperitoneally given zanamivir-DNP conjugate/zanamivir/PBS 24 h post-infection for only one time and mice were counted as dead when losing either 25% of their initial weight or when they were moribund.

As shown in FIG. 39A-B, intraperitoneally administered zanamivir-DNP provides good protection 14 days of post infection (all mice were cured), whereas zanamivir alone does not provide the same protection.

### Example 22. Anti-DNP Antibody Treated Unimmunized Mice Infected by H1N1 Virus (FIG. 40A-B)

In this Example, unimmunized mice were intravenously given anti-DNP antibody one day after infected with lethal dose of H1N1 virus and immediately treated by various doses of intraperitoneally administered zanamivir-DNP conjugate.

In the pilot study, mice (3 mice/group) were infected with 50 uL A/Puerto Rico/8/34 virus (100 LD₅₀, 4.2×10⁵ PFU) at day 0.

Mice were intravenously given anti-DNP antibody (polyclonal rabbit IgG) 24 h post-infection for only one time and intranasally given zanamivir-DNP conjugate 24 h post-infection for only one time.

Mice were counted as dead when losing either 25% of their initial weight or when they were moribund.

As shown in FIG. 40A-B, if concurrently administered 24 hours post infection, as low as 1 mg/kg of intravenously administered anti-DNP antibody along with 1.5 umol/kg intranasally administered zana-DNP conjugate are able to provide necessary protection to lethal dose of H1N1 virus infection.

### Example 23. Synthesis Scheme of Zanamivir-Rhamnose Conjugate (FIG. 41)

In this Example, a different conjugate zanamivir-rhamnose is synthesized to be used in induction of immune response to influenza virus infection. The synthesis scheme is provided in FIG. 41.

### Example 24. Competitive Binding of Zanamivir-Rhamnose Conjugate to Neuraminidase Transfected HEK293 Cells Using Zanamivir-Rhodamine Conjugate as the Labelled Ligand (FIG. 42A-B)

In this Example, Neuraminidase transfected HEK 293 cells were seed at 24 well plates and incubated overnight. In the next day the cells were incubated with a single concentration of labeled ligand (15 nM zanamivir-rhodamine conjugate) as well as with various concentrations of zanamivir-rhamnose conjugate or zanamivir. After incubated for 1 h, the cells were washed with the cell culture medium and the remaining fluorescence was quantitated by fluorescence spectroscopy. Apparent K_{d} was calculated by plotting cell bound fluorescence intensity versus the log concentration of zanamivir-DNP conjugate or zanamivir added using the competition binding equation in GraphPad Prism 4. K_{d} about 3.57 nM is plotted for zanamivir-rhamnose conjugate as compared to free zanamivir K_{d} about 0.77 nM, and zanamivir-rhodamine conjugate K_{d} about 11.71 nM.

### Example 25. Immunotherapy Study with Zanamivir-Rhamnose Conjugate (FIG. 43A-B)

In this Example, mouse protection by zanamivir-rhamnose conjugate is observed in a dose escalation study. Briefly, rhamnose-OVA immunized mice (5 mice/group) were infected with 50 uL A/Puerto Rico/8/34 virus (100 LD⁵⁰, 4.2×10⁵ PFU) at day 0.

Mice were intranasally given 1.5/0.5/0.17 umol/kg zanamivir-rhamnose conjugate/zanamivir/PBS 24 h post-infection, twice daily for 5 days and mice were counted as dead when losing either 25% of their initial weight or when they were moribund.

As shown in FIGS. 43A-B, as little as 0.17 umol/kg zanamivir-rhamnose conjugate b.i.d was able to provide at least 50% protection for A/Puerto Rico/8/34 (H1N1) virus lethally infected mice.

### REFERENCES

1. Organization, W. H. Influenza (Seasonal). Fact Sheet No 211. 2014. In 2014.
2. Control, C. f. D.; Prevention. Estimating seasonal influenza-associated deaths in the United States. Disponibile su: http://www.cdc. gov flui about disease us flu-related deaths. htm 2015.
3. Bush, G. W. The national security strategy of the United States of America. Wordclay: 2009.
4. De Clercq, E. Antiviral agents active against influenza A viruses. Nature reviews Drug discovery 2006, 5, 1015.
5. Control, C. f. D.; Prevention. Influenza antiviral drug resistance: questions & answers. In 2008.
6. Ebell, M. H.; Call, M.; Shinholser, J. Effectiveness of oseltamivir in adults: a meta-analysis of published and unpublished clinical trials. Family practice 2012, 30, 125-133.
7. Von Itzstein, M. The war against influenza: discovery and development of sialidase inhibitors. Nature reviews Drug discovery 2007, 6, 967.

8 Srinivasarao, M.; Galliford, C. V.; Low, P. S. Principles in the design of ligand-targeted cancer therapeutics and imaging agents. Nature reviews Drug discovery 2015, 14, 203.
9. Lamb, R. A.; Krug, R.; Knipe, D. Fields virology. Fields virology 2001, 1.
10. Nayak, D. P.; Balogun, R. A.; Yamada, H.; Zhou, Z. H.; Barman, S. Influenza virus morphogenesis and budding. Virus research 2009, 143, 147-161.
11. Murray, B. C.; Peterson, M. T.; Fecik, R. A. Chemistry and biology of tubulysins: antimitotic tetrapeptides with activity against drug resistant cancers. Natural product reports 2015, 32, 654-662.
12. Clark, M. P.; Ledeboer, M. W.; Davies, I.; Byrn, R. A.; Jones, S. M.; Perola, E.; Tsai, A.; Jacobs, M.; Nti-Addae, K.; Bandarage, U. K. Discovery of a novel, first-in-class, orally bioavailable azaindole inhibitor (VX-787) of influenza PB2. Journal of medicinal chemistry 2014, 57, 6668-6678.
13. Byrn, R. A.; Jones, S. M.; Bennett, H. B.; Bral, C.; Clark, M. P.; Jacobs, M. D.; Kwong, A. D.; Ledeboer, M. W.; Leeman, J. R.; McNeil, C. F. Preclinical activity of VX-787, a first-in-class, orally bioavailable inhibitor of the influenza virus polymerase PB2 subunit. Antimicrobial agents and chemotherapy 2015, 59, 1569-1582.
14. Liu, Q., Zhou, Y.-h.; Yang, Z.-q. The cytokine storm of severe influenza and development of immunomodulatory therapy. Cellular & molecular immunology 2016, 13, 3.
15. Oldstone, M. B.; Teijaro, J. R.; Walsh, K. B.; Rosen, H. Dissecting influenza virus pathogenesis uncovers a novel chemical approach to combat the infection. Virology 2013, 435, 92-101.
16. Scott, F.; Clemons, B.; Brooks, J.; Brahmachary, E.; Powell, R.; Dedman, H.; Desale, H.; Timony, G.; Martinborough, E.; Rosen, H. Ozanimod (RPC1063) is a potent sphingosine-1-phosphate receptor-1 (SIP1) and receptor-5 (S1P5) agonist with autoimmune disease-modifying activity. British journal of pharmacology 2016, 173, 1778-1792.
17. Walsh, K. B., Teijaro, J. R.; Wilker, P. R., Jatzek, A.; Fremgen, D. M.; Das, S. C.; Watanabe, T.; Hatta, M.; Shinya, K.; Suresh, M. Suppression of cytokine storm with a sphingosine analog provides protection against pathogenic influenza virus. Proceedings of the National Academy of Sciences 2011, 108, 12018-12023.
18. Teijaro, J. R.; Walsh, K. B., Cahalan, S.; Fremgen, D. M., Roberts, E.; Scott, F.; Martinborough, E.; Peach, R.; Oldstone, M. B.; Rosen, H. Endothelial cells are central orchestrators of cytokine amplification during influenza virus infection. Cell 2011, 146, 980-991.
19. Pommier, Y. Topoisomerase 1 inhibitors: camptothecins and beyond. Nature Reviews Cancer 2006, 6, 789.
20. Rialdi, A., Campisi, L.; Zhao, N., Lagda, A. C.; Pietzsch, C.; Ho, J. S. Y.; Martinez-Gil, L.; Fenouil, R.; Chen, X., Edwards, M. Topoisomerase 1 inhibition suppresses inflammatory genes and protects from death by inflammation. Science 2016, 352, aad7993.
21. Lu, Y.; You, F.; Vlahov, I.; Westrick, E.; Fan, M.; Low, P. S.; Leamon, C. P. Folate-targeted dinitrophenyl hapten immunotherapy: effect of linker chemistry on antitumor activity and allergic potential. Molecular pharmaceutics 2007, 4, 695-706.
22. Lu, Y.; Sega, E.; Leamon, C. P.; Low, P. S. Folate receptor-targeted immunotherapy of cancer: mechanism and therapeutic potential. Advanced drug delivery reviews 2004, 56, 1161-1176.
23. McEnaney, P. J.; Parker, C. G.; Zhang, A. X. Antibody-Recruiting Small Molecules: Synthetic Constructs as Immunotherapeutics. In Anmial Reports in Medicinal Chemistry, Elsevier: 2017; Vol. 50, pp 481-518.
24. Sheridan, R. T.; Hudon, J.; Hank, J. A.; Sondel, P. M.; Kiessling, L. L. Rhamnose Glycoconjugates for the Recruitment of Endogenous Anti-Carbohydrate Antibodies to Tumor Cells. ChemBioChem 2014, 15, 1393-1398.
25. Wu, X.; Wu, X.; Sun, Q.; Zhang, C.; Yang, S.; Li, L.; Jia, Z. Progress of small molecular inhibitors in the development of anti-influenza virus agents. Theranostics 2017, 7, 826.
26. Honda, T.; Masuda, T.; Yoshida, S.; Arai, M.; Kaneko, S.; Yamashita, M. Synthesis and anti-influenza virus activity of 7-O-alkylated derivatives related to zanamivir. Bioorganic & medicinal chemistry letters 2002, 12, 1925-1928.
27. Xu, X.; Zhu, X.; Dwek, R. A.; Stevens, J.; Wilson, I. A. Structural characterization of the 1918 influenza virus H1N1 neuraminidase. Journal of virology 2008, 82, 10493-10501.
28. Feng, E.; Ye, D.; Li, J.; Zhang, D.; Wang, J.; Zhao, F.; Hilgenfeld, R.; Zheng, M.; Jiang, H.; Liu, H. Recent Advances in Neuraminidase Inhibitor Development as Anti-influenza Drugs. ChemMedChem 2012, 7, 1527-1536.

1. (canceled)

2. A method to treat influenza virus infection in a subject, comprising treating the subject with a conjugate, wherein the conjugate consists of a single targeting ligand, a single linker, and a single hapten, wherein the targeting ligand is a molecule that binds neuraminidase and the linker is covalently bound to the hapten and the targeting ligand.

3. The conjugate according to claim 2, wherein the targeting ligand is selected from the group consisting of oseltamivir, zanamivir, peramivir and laninamivir.

4. The method according to claim 2, wherein the targeting ligand is zanamivir.

5. The method according to claim 2, wherein the hapten is selected from the group consisting of a dinitrophenyl group, a trinitrophenyl group, a rhamnosyl group, and an alpha-galactosyl group.

6. The method according to claim 2, wherein the conjugate has the formula:

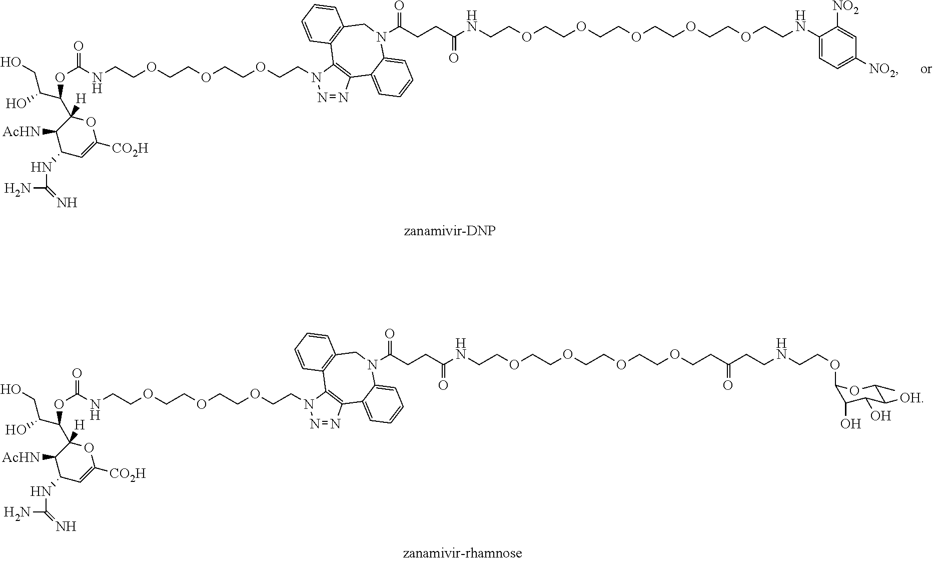 [SMILES: CC(=O)N[C@H]1[C@H]([C@H](OC(=O)NCCOCCOCCOCCn2nnc3c2-c2ccccc2CN(C(=O)CCC(=O)NCCOCCOCCOCCOCCOCCNc2ccc([N+](=O)[O-])cc2[N+](=O)[O-])c2ccccc2-3)[C@H](O)CO)OC(C(=O)O)=C[C@@H]1NC(=N)N ; CC(=O)N[C@H]1[C@H]([C@H](OC(=O)NCCOCCOCCOCCn2nnc3c2-c2ccccc2CN(C(=O)CCC(=O)NCCOCCOCCOCCOCCC(=O)CCNCCOC2OC(C)C(O)C(O)C2O)c2ccccc2-3)[C@H](O)CO)OC(C(=O)O)=C[C@@H]1NC(=N)N]

7. The method according to claim 2, wherein the conjugate elicits immune responses leading to the clearance of antibody-coated virus or virus-infected cells via antibody-dependent cellular phagocytosis (ADCP), antibody-dependent cellular cytotoxicity (ADCC) or complement-dependent cytotoxicity (CDC).

8. The method according to claim 2, wherein the conjugate has a binding affinity to neuraminidase at between about 1 nM to about 15 nM.
